(19) United States
(12) Patent Application Publication
(10) Pub. No.: US 2023/0223103 A1
(43) Pub. Date: Jul. 13, 2023

(54) SYSTEMS AND METHODS FOR INTELLIGENT GENOTYPING BY ALLELES COMBINATION DECONVOLUTION

(71) Applicant: LIFE TECHNOLOGIES CORPORATION, Carlsbad, CA (US)

(72) Inventor: Chantal Roth, (US)

(73) Assignee: LIFE TECHNOLOGIES CORPORATION, Carlsbad, CA (US)

(21) Appl. No.: 18/090,392

(22) Filed: Dec. 28, 2022

Related U.S. Application Data
(60) Provisional application No. 63/294,342, filed on Dec. 28, 2021.
(60) Provisional application No. 63/330,287, filed on Apr. 12, 2022.

(51) Int. Cl. G16B 20/00; G16B 40/20

(52) U.S. Cl. CPC G16B 20/00; G16B 40/20

(57) ABSTRACT

Methods and systems for improving computer efficiency by intelligently selecting subsets of possible short tandem repeat (STR) allele combinations for further deconvolution analysis are disclosed. In one embodiment, at each locus, for a currently analyzed contribution ratio scenario of a plurality of contribution ratio scenarios, a processor computes an adjusted evidence profile. For a first, or next, unidentified contributor having a pre-determined highest remaining contribution ratio in the currently analyzed contribution ratio scenario for the plurality of contributors, a processor computes a first range of expected peak heights using at least the pre-determined highest remaining contribution ratio, a selected degradation value, and a peak height ratio distribution. Also disclosed are methods and systems for intelligently estimating the number of contributors to a biological sample.

### CROSS-REFERENCE TO RELATED APPLICATIONS

This application claims the benefit of U.S. Provisional Application Ser. No. 63/294,342, filed on Dec. 28, 2021 and of U.S. Provisional Application No. 63/330,287 filed on Apr. 12, 2022. To the extent permitted in applicable jurisdictions, the entire contents of these applications are incorporated herein by reference.

### BACKGROUND

The present disclosure relates generally to genotyping, and more specifically to systems, devices, and methods for deconvolution analysis of biological samples potentially having multiple contributors.

Genotyping is the process of determining, from a biological sample, what combination of alleles (particular DNA sequence variations) individuals have at a particular genetic locus. Human identification, e.g., for forensic purposes, is typically carried out by analyzing alleles at several known short tandem repeat (STR) loci. STR regions have short repeated sequences of DNA. The most common is a repeating sequence (“repeat unit”) of four bases. But some STR loci have repeat units of different lengths, e.g., in the range of 2-7 bases. Different STR alleles at a particular locus have different numbers of repeat units at that locus. STR loci have significant variability across individuals. Although two different individuals might have the same genotype (i.e., the same combination of two alleles) at a given STR locus, when several STR loci are considered, the likelihood of two individuals have the exact same genotype at each STR locus is extraordinarily small, i.e., very close to zero. Thus, analyzing genotypes at several STR loci is a reliable way of identifying individuals from biological samples.

In forensic investigations, thirteen core STR loci are routinely used for DNA profiling. The 13 core STR loci include, for example, TPOX, VWA, AMEL, TH01, FGA, D3S1358, and others. These STR loci are targeted with sequence-specific primers and fragments corresponding to the loci are amplified using PCR. The DNA fragments that result are then separated and detected using electrophoresis. There are two common methods of separation and detection, capillary electrophoresis (CE) and gel electrophoresis, with CE being more common in recent years. Next generation sequencing technologies have also been recently used for DNA profiling.

### SUMMARY

In forensic investigations, a biological sample often contains DNA from multiple contributors. The identity of some or all of these contributors may be unknown. Moreover, the proportion of the sample attributed to each contributor is not necessarily known either. Frequently, the exact number of contributors of a given biological sample is also unknown. The process of determining which contributors have what genotypes in a multiple-contributor sample is known as deconvolution analysis. Before the deconvolution analysis can be performed, the number of contributors of a sample often needs to be assumed or estimated.

Existing deconvolution techniques typically compute and analyze theoretical profiles against the evidence profile for each and every conceivable combination of alleles at a given locus. But these techniques are time consuming, and it is also not necessary and not efficient to go through every conceivable scenario. Thus, there is a need for more intelligent deconvolution analysis systems and methods.

Embodiments of the present invention improve computer efficiency by intelligently selecting subsets of possible short tandem repeat (STR) allele combinations for further deconvolution analysis using an evidence profile obtained from the biological sample and expected signal ranges for unidentified contributors.

In some embodiments of the invention, at each locus, for a currently analyzed contribution ratio scenario of a plurality of contribution ratio scenarios, a processor computes an adjusted evidence profile by subtracting from the evidence profile a computed expected contribution of all known contributors, if any. For a first, or next, unidentified contributor having a pre-determined highest remaining contribution ratio in the currently analyzed contribution ratio scenario for the plurality of contributors, a processor computes a first range of expected peak heights using at least the pre-determined highest remaining contribution ratio, a selected degradation value, and a peak height ratio distribution.

In some embodiments, for all other remaining unidentified contributors, if any, a processor computes a second range of expected peak heights using at least pre-determined contribution ratios in the currently analyzed contribution ratio scenario of all other remaining unidentified contributors, the selected degradation value, and the peak height ratio distributions. The processor further uses the adjusted evidence profile and one or more of the first range and the second range to select one or more selected genotypes corresponding to the first, or next, unidentified contributor for further deconvolution analysis. The one or more selected genotypes comprises fewer genotypes than a total number of genotypes potentially associated with a current locus in a general population.

In some embodiments of the present disclosure, improved methods and systems for estimating the number of contributors (NOC) to a biological sample are provided. In some embodiments, improved NOC analysis techniques for estimating NOC can enhance deconvolution analysis, making it more tractable, faster, and efficient deconvolution analysis. Existing techniques of estimating the number of contributors (NOC) of a biological sample uses a single model to provide a prediction or simply assumes all possible numbers of contributors. But these techniques may not be accurate under all situations and/or may be very time consuming if all possible numbers of contributors are being considered. For example, by considering all possible numbers of contributors of a sample, the deconvolution analysis may need to compute many possible scenarios for each possible number of contributors. Therefore, if all possible numbers of contributors are considered, the computational effort of the deconvolution analysis may be prohibitively or impractically large and time consuming. It is also unnecessary and inefficient to consider every conceivable scenario of the number of contributors. Thus, there is a need for more intelligent NOC prediction systems and methods.

Embodiments of the present invention improve computer efficiency by intelligently predicting the number of contributors of a biological sample using a combination of multiple models, including one or more machine-learning based models and/or one or more non-machine learning based models. The combination of models may be pre-selected based on past analysis results, user preferences, the accuracy of each model, or the like. Weights are assigned to each of the selected models so that a more accurate prediction of the NOC can be achieved.

In some embodiments of the invention, a method for predicting the number of contributors associated with a biological sample for enhancing computer efficiency in genotyping at least one contributor using an evidence profile obtained from the biological sample comprising genetic signal data are provided. The method comprises using one or more computer processors to carry out processing comprising receiving an indication of a combination of selected models for estimating the number of contributors associated with the biological sample and generating a predetermined number of computed sample profiles based on the evidence profile. The method further comprises, for each selected model of the combination of selected models, estimating probabilities of a plurality of number-of-contributors possibilities using the selected model and the computed sample profiles and predicting the number of contributors using the estimated probabilities of the plurality of NOC possibilities obtained based on the combination of selected models.

In some embodiments, a computer-implemented method of predicting the number of contributors associated with a biological sample for enhancing computer efficiency in genotyping at least one contributor using an evidence profile obtained from the biological sample comprising genetic signal data is provided. The method comprising using one or more computer processors to carry out processing comprising: receiving an expected maximum number of contributors and generating a predetermined number of computed sample profiles based on the evidence profile and the expected maximum number of contributors. The method further comprises, for each possible number of contributors that is less than or equal to the expected maximum number of contributors, determining a number of peaks for each computed sample profile having a corresponding possible number of contributors; obtaining an expected peak count distribution based on the number of peaks for each computed sample profile having a corresponding possible number of contributors; determining a total number of peaks in the evidence profile; and estimating, based on the expected peak count distribution and the total number of peaks in the evidence profile, the probabilities of the plurality of NOC possibilities.

These and other embodiments are described more fully below.

### BRIEF DESCRIPTION OF THE DRAWINGS

FIG. 1 illustrates an example of a capillary electrophoresis sequencing system in accordance with an embodiment of the present disclosure;

FIG. 2A illustrates an example of a biological sample having multiple contributors and a corresponding evidence profile generated by analyzing the biological sample in accordance with some embodiments of the present disclosure;

FIG. 2B illustrates an example of a biological sample and a corresponding evidence profile generated by analyzing the biological sample in accordance with an embodiment of the present disclosure;

FIG. 3A is a high-level flowchart illustrating a method of performing a NOC prediction in accordance with some embodiments of the present disclosure;

FIG. 3B is a high-level flowchart illustrating a method of performing a deconvolution analysis in accordance with an embodiment of the present disclosure;

FIG. 4 illustrates example scenarios used in forensic investigations in accordance with an embodiment of the present disclosure;

FIG. 5 is a flowchart illustrating a method of a deconvolution analysis in accordance with an embodiment of the present disclosure;

FIG. 6 is a flowchart illustrating a method of computing a first range of expected peak heights and a second range of expected peak heights for computerized display of an adjusted evidence profile in accordance with an embodiment of the present disclosure;

FIG. 7A is a flowchart illustrating a method of determining selected subset of allele combinations for a currently analyzed unknown contributor in accordance with an embodiment of the present disclosure;

FIGS. 7B-7G illustrate various scenarios of selecting allele combinations in accordance with an embodiment of the present disclosure;

FIG. 8 is a flowchart illustrating a method for computing theoretical contributions in accordance with an embodiment of the present disclosure;

FIG. 9 is a flowchart illustrating a method for computing a theoretical profile for an allele combination of a currently analyzed contribution ratio at a locus in accordance with an embodiment of the present disclosure;

FIG. 10 is a flowchart illustrating a method for computing a score of a bin in a theoretical profile in accordance with one embodiment of the present disclosure;

FIG. 11 illustrates scenarios of comparing a theoretical profile with an evidence profile in accordance with one embodiment of the present disclosure; and

FIG. 12 illustrates an example of a user interface for displaying scores of multiple theoretical profiles in accordance with one embodiment of the present disclosure;

FIG. 13 illustrates an example user interface for receiving user inputs and displaying deconvolution analysis parameters and results in accordance with one embodiment of the present disclosure;

FIG. 14 is a flowchart illustrating a method of predicting the number of contributors of a sample in accordance with some embodiments of the present disclosure;

FIG. 15 is a flowchart illustrating a method of generating a predetermined number of computed sample profiles in accordance with some embodiments of the present disclosure;

FIG. 16A is a flowchart illustrating a method of determining features of peak height distribution for each computed sample profile in accordance with some embodiments of the present disclosure;

FIG. 16B is an example computed sample profile generated by using methods described in the present disclosure;

FIG. 17A illustrates histogram plots for all loci of computed sample profiles having one contributor in accordance with some embodiments of the present disclosure;

FIG. 17B illustrates histogram plots for all loci of computed sample profiles having two contributors in accordance with some embodiments of the present disclosure;

FIG. 18 is a flowchart illustrating a method for determining one or more other features for each computed sample profile in accordance with some embodiments of the present disclosure;

FIG. 19 illustrates an expected peak count distribution in accordance with some embodiments of the present disclosure;

FIGS. 20A-20B illustrate a computer interface for visualizing impact on peak count distributions of different selections of minimum relative fluorescent unit (RFU) for counting peaks in accordance with some embodiments of the present disclosure.

FIGS. 21A-21B illustrate a computer interface for visualizing the relationship between peak height minimums (minimum RFU) and maximum allele counts (MAC) from analyzed samples in accordance with some embodiments of the present disclosure.

FIG. 22 illustrates a block diagram of an example computing device that may incorporate embodiments of the present disclosure.

While embodiments of the disclosure are described with reference to the above drawings, the drawings are intended to be illustrative, and other embodiments are consistent with the spirit, and within the scope, of the disclosure.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

The number of repeats at a short tandem repeat (STR) locus is referred to as an allele. For example, the STR locus known as D7S820, found on chromosome 7, contains between five and sixteen repeats of GATA. Therefore, twelve different alleles are possible for the D7S820 STR locus. An individual with D7S820 alleles 10 and 15, for example, would have inherited a copy of D7S820 with 10 GATA repeats from one parent, and a copy of D7S820 with 15 GATA repeats from the other parent. Because there are 12 different alleles for this STR locus, there are therefore 78 different possible genotypes. A genotype corresponds to a pair of alleles. Specifically, there are 12 homozygotes, in which the same allele is received from each parent (e.g., (5, 5), (6, 6), (7, 7), or the like). And there are 66 heterozygotes, in which the two alleles received from the parents are different in a genotype (e.g., (5, 6), (7, 10), (11, 15), or the like).

In genotyping for forensic investigations, a biological sample collected from a crime scene often includes contributions from multiple contributors (e.g., a suspect, a victim, a person who discovered the crime scene, etc.). Many variables potentially explain a particular evidence profile including, for example, the number of contributors, genotypes of those contributors, a sample degradation level, relative proportions (ratios) of contributions from the each of the contributors, lab equipment variations, etc.

In conventional approaches, genotyping mixed samples by accounting for potential variables typically requires a massive number of simulations, using, for example, Markov Chain Monte Carlo techniques to generate millions of random profiles for comparison. This process may take a very long time, e.g., days, weeks, or even months, to complete and thus is very inefficient. Moreover, if the Monte Carlo simulations do not converge, the simulations will fail to provide any meaningful results. The simulations may then need to be repeated, which causes further delay, increases energy consumption, and reduces analysis efficiency. The delay is sometimes significant and intolerable. The methods described herein intelligently select allele combinations for deconvolution analysis and thus improve timeliness and efficiency relative to prior art technologies.

Embodiments of the present disclosure discussed herein intelligently select only limited subsets of allele combinations for deconvolution analysis. This significantly reduces the number of allele combination that need to be subjected to full deconvolution analysis relative to prior art methods. Therefore, results can be obtained much faster without sacrificing accuracy. The embodiments described herein thus improve the efficiency of computer-implemented genotyping.

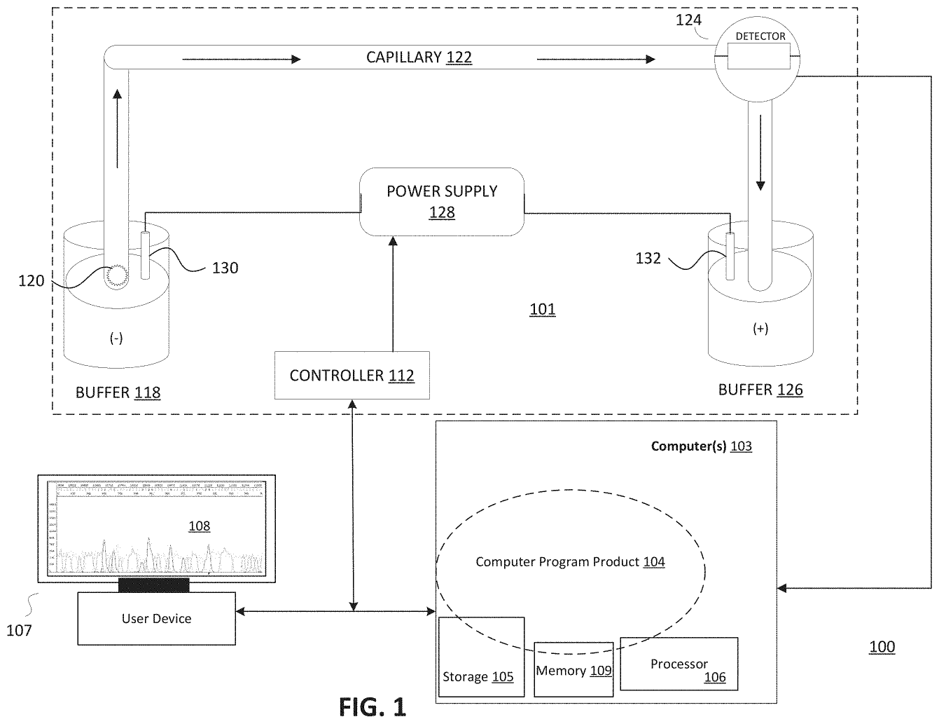

FIG. 1 illustrates system 100 in accordance with an example embodiment of the present disclosure. System 100 comprises capillary electrophoresis (“CE”) instrument 101, one or more computers 103, and user device 107. System 100 can be used to provide sequencing data or evidence profile from a biological sample.

Referencing FIG. 1, a CE instrument 101 in one embodiment comprises a source buffer 118 containing buffer and receiving a fluorescently labeled sample 120, a capillary 122, a destination buffer 126, a power supply 128, and a controller 112. Sample 120 may be a biological sample collected for genotyping with fluorescence labels added. Sample 120 may thus include biological materials from one or more contributors, known or unknown. The source buffer 118 is in fluid communication with the destination buffer 126 by way of the capillary 122. The power supply 128 applies voltage to the source buffer 118 and the destination buffer 126 generating a voltage bias through an anode 130 in the source buffer 118 and a cathode 132 in the destination buffer 126. The voltage applied by the power supply 128 is configured by a controller 112 operated by the computing device 103. The fluorescently labeled sample 120 near the source buffer 118 is pulled through the capillary 122 by the voltage gradient, and optically labeled nucleotides of the DNA fragments within the sample are detected as they pass through an optical sensor 124 on the way to destination buffer 126. Differently sized DNA fragments within the fluorescently labeled sample 120 are pulled through the capillary at different times due to their size.

The optical sensor 124 detects the fluorescent labels on the nucleotides as an image signal and communicates the image signal to the computing device 103. The computing device 103 aggregates the image signal as sample data and generates an electropherogram that may be shown on a display 108 of user device 107. The electropherogram includes a DNA profile with peaks and their corresponding allele numbers. The electropherogram can include, for example, an evidence profile of a biological sample collected from a crime scene.

Instructions for implementing relevant processing reside on computing device 103 in computer program product 104 which is stored in storage 105 and those instructions are executable by processor 106. When processor 106 is executing the instructions of computer program product 104, the instructions, or a portion thereof, are typically loaded into working memory 109 from which the instructions are readily accessed by processor 106. In one embodiment, computer program product 104 is stored in storage 105 or another non-transitory computer readable medium (which may include being distributed across media on different devices and different locations). In alternative embodiments, the storage medium is transitory.

In one embodiment, processor 106 comprises multiple processors which may comprise additional working memories (additional processors and memories not individually illustrated) including a graphics processing unit (GPU) comprising at least thousands of arithmetic logic units supporting parallel computations on a large scale. Other embodiments comprise one or more specialized processing units comprising systolic arrays and/or other hardware arrangements that support efficient parallel processing. In some embodiments, such specialized hardware works in conjunction with a CPU and/or GPU to carry out the various processing described herein. In some embodiments, such specialized hardware comprises application specific integrated circuits and the like (which may refer to a portion of an integrated circuit that is application-specific), field programmable gate arrays and the like, or combinations thereof. In some embodiments, however, a processor such as processor 106 may be implemented as one or more general purpose processors (preferably having multiple cores) without necessarily departing from the spirit and scope of the present disclosure.

User device 107 incudes a display 108 for displaying results of processing carried out on computers 103.

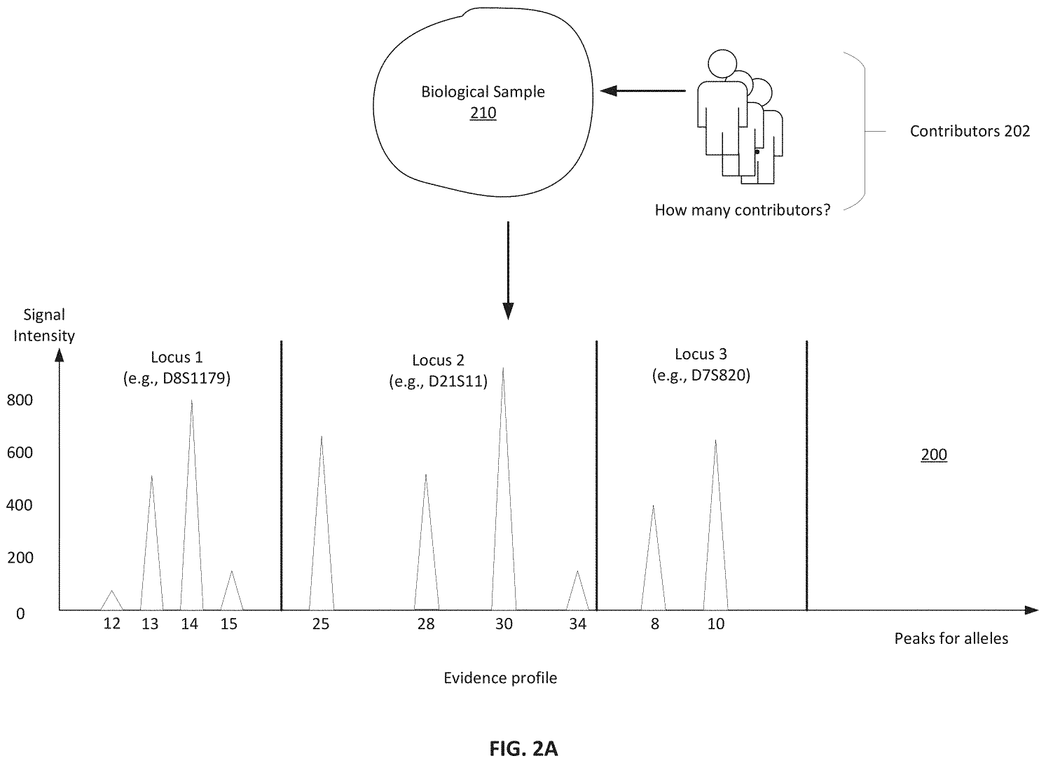

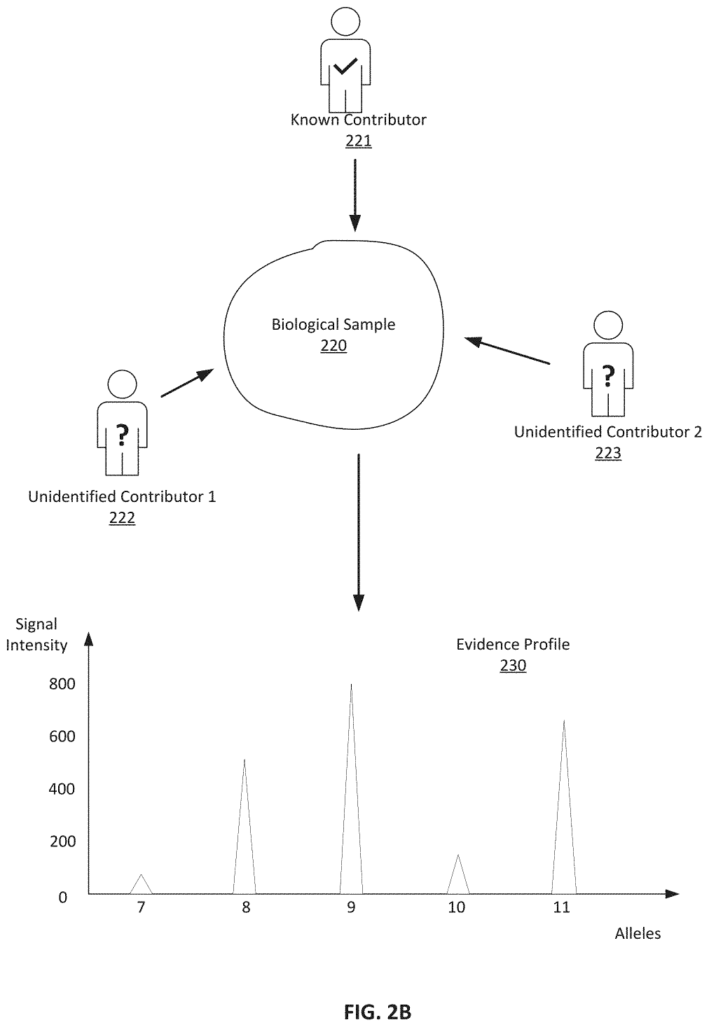

FIG. 2A illustrates an example of a biological sample 210 and a corresponding evidence profile 200 generated by analyzing biological sample 210 using, for example, CE instrument 101 shown in FIG. 1. As shown in FIG. 2, biological sample 210 comprises genetic materials from multiple contributors such as contributors 202. The identities of some contributors may be known. But others may be unknown. Therefore, the total number of the contributors 202 maybe uncertain. For example, while one of the contributors 202 may be ascertained to be the victim, the number of other possible contributors and their identities may be unknown. Moreover, biological sample 210 has a mixture of genetic materials contributed by each of the contributors 202 and their proportions of contributions may also be unknown. Therefore, before performing any analysis to explain the evidence profile, the number of contributors of the biological sample needs to be predicted.

In FIG. 2A, evidence profile 200, obtained from analyzing biological sample 210, includes a graph with the y-axis representing the signal intensity in relative fluorescence units (“RFU”) and the x-axis representing the allele number at different loci. As described above, in forensic analysis, analyzing alleles at several known short tandem repeat (STR) loci facilitates identification of contributors. The number of repeats at a short tandem repeat (STR) locus is referred to as an allele. For example, the STR locus known as D7S820, found on chromosome 7, contains between five and sixteen repeats of GATA. Therefore, excluding partial repeats in the form of microvariants, twelve different alleles are possible for the D7S820 STR locus. An individual with D7S820 alleles 10 and 15, for example, would have inherited a copy of D7S820 with 10 GATA repeats from one parent, and a copy of D7S820 with 15 GATA repeats from the other parent. Because there are 12 different alleles for this STR locus, there are therefore 78 different possible genotypes. A genotype corresponds to a pair of alleles. Specifically, there are 12 homozygotes, in which the same allele is received from each parent (e.g., (5, 5), (6, 6), (7, 7), or the like). And there are 66 heterozygotes, in which the two alleles received from the parents are different in a genotype (e.g., (5, 6), (7, 10), (11, 15), or the like).

Evidence profile 200 represents signal intensities of detected fluorescent labels on the nucleotides as a sequence of peaks corresponding to different alleles (e.g., 7-11, 12-15) at different loci. The signals corresponding to the fluorescently labelled nucleotides may be displayed in different colors, in grayscale, or as different variations of black and white hatched lines representing the various colors. As shown in FIG. 2A, evidence profile 200 includes, for example, peaks associated with several loci such as locus 1 (e.g., D8S1179), locus 2 (e.g., D21S11), locus 3 (D7S820), or the like. Each peak has its peak height. In CE, the peak height of an allele peak refers to the signal intensity of a signal (e.g., 500 RFU) for a particular allele at a particular locus. For next generation sequencing (NGS) techniques used to detect alleles at STR loci and/or to detect SNP (single nucleotide polymorphisms), the peak height refers to the number of reads at a particular locus at a particular genomic position for a particular allele or SNP. For NGS techniques used to detect microhaplotypes (MH), the peak height refers to the number of reads that cover an entire phased allele for a particular locus or target (e.g., the number of reads that make up the allele genome build and allele definition conversion tool, or GACT). Microhaplotype loci are a type of molecular marker of less than 300 nucleotides, defined by two or more closely linked SNPs associated in multiple allelic combinations. These markers facilitate massively parallel sequencing analysis using the NGS techniques.

As described above, evidence profile 200 can be obtained by analyzing biological sample 210 using, for example, CE instrument 101. Because biological sample 210 contains biological materials contributed by multiple contributors, evidence profile 200 is a mixed profile and deconvolution analysis needs to be performed to assign DNA profiles (genotypes at each locus) to the different contributors that explain the peaks in evidence profile 200 with sufficiently high certainty. Oftentimes, to perform such a deconvolution analysis, the number of contributors needs to be predicted beforehand.

It is understood that evidence profile 200 shown in FIG. 2A is a simplified profile for illustration purposes. An actual evidence profile may include more or fewer loci, different loci, more or fewer peaks, different peaks, various stutter peaks, noise etc. Stutter peaks are small peaks that typically occur one repeat unit before or after a real peak. During the PCR amplification process, the polymerase can lose its place when copying a strand of DNA, e.g., slipping forwards or backwards for a number of base pairs, thereby causing stutter peaks.

FIG. 2B illustrates an example of a biological sample 220 and a corresponding evidence profile 230 generated by analyzing biological sample 220. As shown in FIG. 2B, biological sample 220 comprises genetic materials from multiple contributors including contributors 221, 222, and 223. In the example shown in FIG. 2B, contributor 221 is a contributor whose identity is known and whose DNA profile is available. Contributors 222 and 223 are contributors whose identities and DNA profiles are unknown. Biological sample 220 has a mixture of biological materials contributed by each of the contributors 221, 222, and 223. In some instances, the exact number of contributors of a biological sample may be predetermined.

In some instances, the number of contributors is ascertained or estimated, but the ratio or proportion of the biological material contributions to the biological sample 220 by each contributor may be unknown. Thus, different scenarios of the contribution ratios can be created and used for a deconvolution analysis. A deconvolution analysis refers to a genotyping analysis that deconvolutes a mixed DNA profile to explain the peaks in the DNA profile and assign an individual profile to each individual contributor.

In some instances, the deconvolution analysis is a partial one, which, for example, does not account for the contribution ratio. By taking into the contributor ratios into account (and optionally the sample degradation level), a full deconvolution analysis can be performed for a mixed DNA evidence profile. A full deconvolution analysis can determine, for example, the actual or most-likely contribution ratios, the actual or most-likely sample degradation ratio, and the actual or most-likely DNA profiles of each contributor. Intelligent genotyping methods used for full deconvolution analysis are described below in more detail.

In FIG. 2B, evidence profile 230, obtained from biological sample 220, includes a graph with the y-axis representing the signal intensity in RFU and the x-axis representing the allele number. Evidence profile 230 represents signal intensities of detected fluorescent labels on the nucleotides as a sequence of peaks corresponding to different alleles, e.g., 7-11. The signals corresponding to the fluorescently labelled nucleotides may be displayed in different colors, in grayscale, or as different variations of black and white hatched lines representing the various colors.

As described above, evidence profile 230 can be obtained by analyzing biological sample 210 using, for example, CE instrument 101. Because biological sample 220 contains biological materials contributed by different contributors, evidence profile 230 is a mixed profile and deconvolution analysis needs to be performed to assign DNA profiles (genotypes at each locus) to the different contributors that explain the peaks in evidence profile 230 with sufficiently high certainty.

It is understood that evidence profile 230 shown in FIG. 2B is a simplified profile for illustration purposes. An actual evidence profile may include more or fewer peaks, various stutter peaks, noise etc. Stutter peaks are small peaks that occur immediately before or after a real peak. During the PCT amplification process, the polymerase can lose its place when copying a strand of DNA, e.g., slipping forwards or backwards for a number of base pairs, thereby causing stutter peaks.

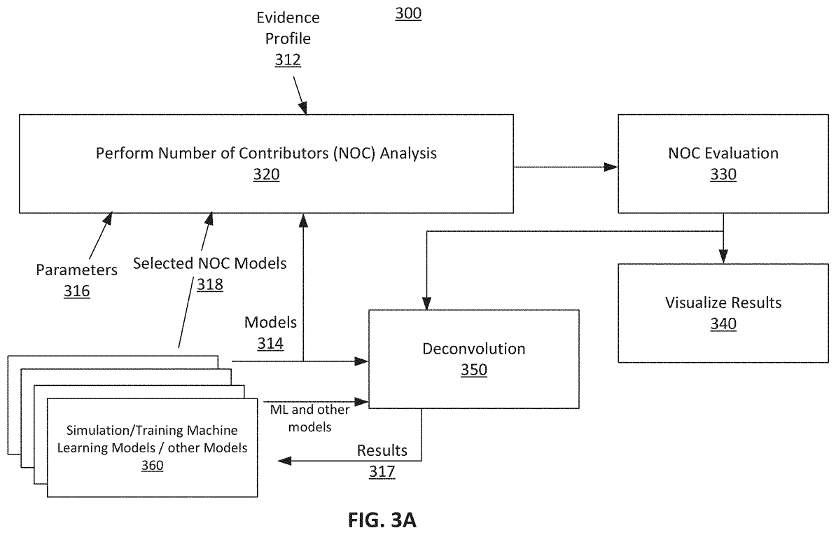

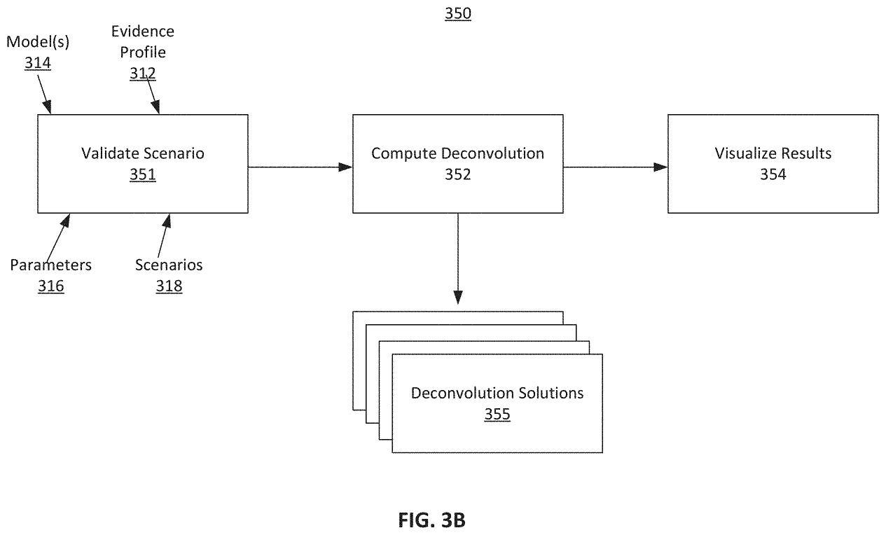

FIG. 3A is a flowchart 300 illustrating a method of predicting the number of contributors. In step 320 shown in FIG. 3, various inputs are provided for estimating the number of contributors using multiple selected models. Such inputs include the evidence profile (or a data representation thereof) 312, statistical models 314, parameters 316, and selected NOC models 318. Evidence profile 312 is a mixed DNA profile obtained from a biological sample having multiple contributors. One example of evidence profile 312 is evidence profile 220 as described above. Model(s) 314 provide one or more distributions related to computing stutter peaks, noise, shoulders, inter locus balance (ILB), peak height imbalance or peak height ratio (PHR), population frequencies and decay, or the like. Model(s) 314 are often needed for generating computed sample profiles. For example, model(s) 314 may include a stutter model, a PHR model, an ILB model, a noise model, population statistics, and a model builder/editor. The model builder/editor allows a user to generate a new model or modify/update an existing model. These distributions or models are typically experimental, empirical, and/or statistical. They may be independent from the biological samples or evidence profiles being analyzed and may be applied among different samples or profiles. In some embodiments, some model(s) 314 may be related to or applicable to a particular biological sample or evidence profile, but not others. In some embodiments, a user may select which model to apply in a particular NOC analysis of an evidence profile and/or may customize certain models for a better NOC analysis.

Parameters 316 may include any variables and/or user-controllable inputs that are used in the NOC analysis. For instance, parameters 316 may include an expected maximum number of contributors, parameters associated with a testing kit, weights assigned to each of the selected NOC models 318, and parameters associated with a testing machine used for obtaining the evidence profile, etc. Some of these parameters are described in more detail below.

The NOC analysis is performed by using a combination of multiple selected NOC models 318. In some embodiments, a user interface listing the available NOC models are provided to the user for selecting NOC models. For example, the user interface can display a checkbox in front of each available NOC model. The user can thus select the desired NOC models by clicking on the corresponding checkboxes. In other embodiments, a default group of multiple NOC models are provided. The user may simply use the default group of multiple NOC models for the NOC analysis or may customize the default group by removing certain models and/or adding other models. The user interface may also provide the capability of adding new models to the list of available models and/or removing existing models from the list of available models. In some embodiments, the NOC models may be automatically selected without user intervention based on, for example, a default setting, past experiment results, predefined policies, etc.

In some embodiments, the list of available NOC models includes, for example, an artificial neural network (ANN) based model, a decision tree based model, a random-forest algorithm based model, and a peak count distribution model. An ANN is a computing network or system that can be trained to perform NOC prediction. As described below in more detail, training of the ANN can be done using features of a large quantity of computed sample profiles (e.g., simulated sample profiles that resemble the evidence profile) and/or real biological sample profiles. An ANN includes a collection of artificial neurons. An artificial neuron receives a signal and processes it. An artificial neuron can communicate signals to neurons that are connected to it. The signal at a connection can be a real number, and the output of each neuron is computed by some non-linear function of the sum of its inputs. The connections between the neurons are also referred to as edges. Neurons and edges typically have associated weights that adjust as the network is being trained. The weights may increase or decrease the strength of the signals at the edges. Neurons may have a threshold such that a signal is sent only if the aggregate signal crosses that threshold. Typically, neurons are aggregated into layers. Different layers may perform different transformations on their inputs. Signals in an ANN can travel from the first layer (the input layer) to the last layer (the output layer) through the middle layers (the hidden layers). Features extracted from a large quantity of computed sample profiles can be used to train, test, and validate a particular ANN model. The trained ANN model can then be provided with features extracted from the evidence profile and generate a NOC prediction based on probabilities of various possible NOCs. Some examples of ANN include supervised neural networks (e.g., a convolutional neural network (CNN), a long short-term memory (LSTM) network, or the like) and/or unsupervised neural networks (e.g., generative adversarial network (GAN) or the like). It is understood that the ANN may be any type of desired neural network that has any number of desired layers or neurons.

A decision tree based model uses a decision tree as a predictive model to predict the number of contributors. A decision tree based model predicts the value of a target variable (e.g., the NOC) based on several input variables. Similar to the ANN model, training of the decision tree based model can be done by using features of a large quantity of computed sample profiles (e.g., simulated sample profiles that resemble the evidence profile) and/or real biological sample profiles. A decision tree can be used, for example, for classification. In a decision tree based model used for classification, all of the input variables (e.g., input features extracted from the computed sample profiles) can have finite discrete domains and there is a single target variable (e.g., target feature) referred to as the classification. Each element of the domain of the classification is referred to as a class. A decision tree or a classification tree is a tree in which each internal (non-leaf) node is labeled with an input feature. The arcs coming from a node labeled with an input feature are labeled with each of the possible values of the target feature or the arc leads to a subordinate decision node on a different input feature. Each leaf of the tree is labeled with a class or a probability distribution over the classes, signifying that the data set has been classified by the tree into either a specific class, or into a particular probability distribution (which, if the decision tree is well-constructed, is skewed towards certain subsets of classes).

A decision tree can be constructed by splitting the source set, constituting the root node of the tree, into subsets-which constitute the successor children. The splitting is based on a set of splitting rules based on classification features. This process is repeated on each derived subset in a recursive manner referred to as recursive partitioning. The recursion is completed when the subset at a node has all the same values of the target variable, or when splitting no longer adds value to the predictions. This process of top-down induction of decision trees is an example of a greedy algorithm. A decision tree can be a classification tree as discussed above, a regression tree, a classification and regression tree (CART), a boosted tree, a bootstrap aggregated (or bagged) decision tree, a rotation forest tree, or the like. In some embodiments, selected features of a large quantity of computed sample profiles can be used to train, test, and validate a particular decision tree based model. The trained decision tree based model can then be provided with features extracted from the evidence profile and generate a NOC prediction (e.g., probabilities of various possible NOCs) as the target variable.

A random forest is a type of the bootstrap aggregating decision tree. Random forests or random decision forests are an ensemble learning method for classification, regression, and other tasks that operates by constructing a multitude of decision trees at training time. For classification tasks, the output of the random forest is the class selected by most trees. For regression tasks, the mean or average prediction of the individual trees is provided. Random decision forests may correct or improve the decision trees' tendency of overfitting to their training set.

The ANN model, the decision tree model, and random forest model described above are all examples of machine-learning based models, for which training and/or training update is needed for the model to provide an accurate prediction of the NOC. In FIG. 3A, selected NOC models 318 may also include non-machine learning based model. An example of a non-machine learning based model is a peak distribution model, which computes a peak count distribution using a large quantity of computed sample profiles and/or real sample profiles. The peak count distribution model is described in more detail below.

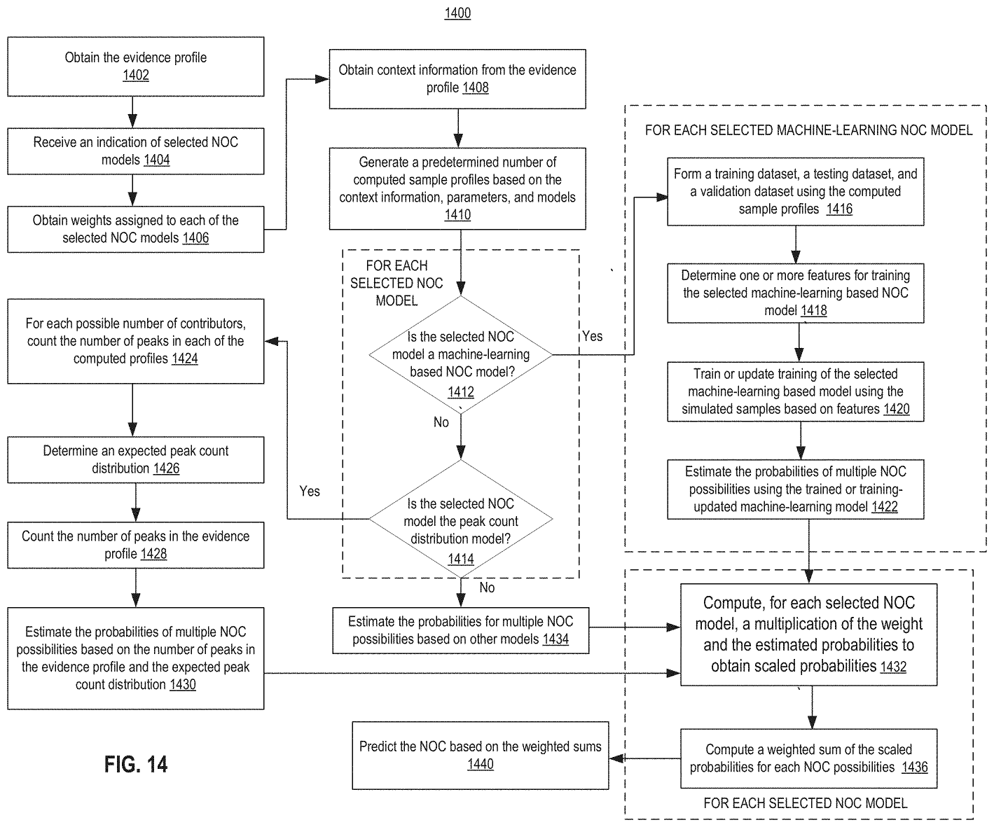

With reference still to FIG. 3A, in step 320, the NOC analysis is performed using a combination of selected NOC models based on the evidence profile 312, models 314, and parameters 316. The analysis results (e.g., in the form of probabilities of possible NOCs) of different NOC models are generated and provided for performing the NOC evaluation in step 330. NOC evaluation is often needed because multiple NOC models may provide different NOC estimations. In some embodiments, the NOC evaluation can be performed using assigned weights to different NOC models. Assigned weights for the selected NOC models are provided (e.g., in parameters 316) based on the user input, default settings, past experimental results, predefined policies, etc. Weights can be used to scale the analysis results of selected NOC models to obtain scaled analysis results. The NOC evaluation process 330 provides a NOC prediction based on the scaled analysis results associated with the multiple selected NOC models. The predicted NOC can be visualized in step 340 (e.g., displayed to the user as a table, graph, chart, etc.). The NOC analysis step 320 and NOC evaluation step 330 are described in greater detail below in the context of FIGS. 14-19. In some embodiments, the predicted NOC is provided to downstream processes for performing, for example, deconvolution analysis 350. Results 317 of deconvolution analysis 350 may be provided to processing 360 for use in training machine learning models and/or for simulations.

FIG. 3B is a flowchart 300 illustrating a method of performing a deconvolution analysis. In step 351 shown in FIG. 3B, various inputs are provided for performing a deconvolution analysis. Such inputs include the evidence profile (or a data representation thereof) 312, one or more model(s) 314, parameters 316, and scenarios 318 that include combinations of hypotheses used in the deconvolution analysis. Evidence profile 312 is a mixed DNA profile corresponding to profiles of several contributors. Evidence profile 312 can be, for example, evidence profile 230 described above. One or more model(s) 314 provide one or more distributions related to computing stutter peaks, noise, shoulders, inter locus balance (ILB), peak height imbalance or peak height ratio (PHR), population frequencies and decay, or the like. For example, model(s) 314 may include a stutter model, a PHR model, an ILB model, a noise model, population statistics, and a model maker. The model maker allows a user to generate a new model or modify/update an existing model. These distributions or models are typically experimental, empirical, or statistical. They may be independent from the biological samples or evidence profiles being analyzed and may be applied among different samples or profiles. In some embodiments, a user may select which model to apply in a particular deconvolution analysis and/or may customize certain models for a better deconvolution analysis.

Parameters 316 may include any variables or user-controllable inputs that are used in the deconvolution analysis. For instance, parameters 316 may include the number of contributors, contribution ratio combinations, sample degradation ratios, loci under analysis, etc. Some of these parameters are described in more detail below.

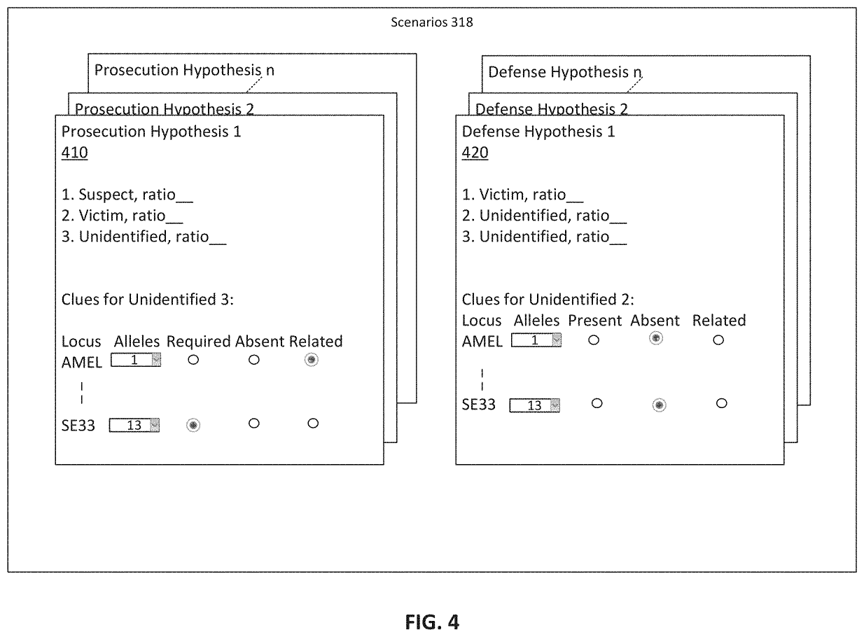

FIG. 4 illustrates scenarios 318 in further detail. Scenarios 318 refer to combinations of hypotheses used in the deconvolution analysis. For example, as shown in FIG. 4, in forensic investigations, a scenario may include a prosecution hypothesis 1 410 and a defense hypothesis 1 420. As an example, prosecution hypothesis 1 410 may indicate that there are three contributors including a suspect, a victim, and an unidentified contributor. The prosecution hypothesis may also indicate the three contributors' respective contribution ratios. Defense hypothesis 1 420 may indicate that there are three contributors including the victim and two unidentified contributors. The defense hypothesis may also indicate respective contribution ratios of the three contributors. In some cases, scenarios 318 may include complex scenarios. As shown in FIG. 4, scenarios 318 may include multiple prosecution hypotheses 1-n and multiple defense hypotheses 1-n.

In some embodiments, a hypothesis can also have clues known or assumed to be true about the unidentified contributors. For example, for unidentified contributor #3 in prosecution hypothesis 1 410, a clue may be that this person has allele 13 at locus SE33. This allele is thus required to be added to an allele combination of a selected genotype used in the deconvolution analysis. Such clues can be helpful to limit the number of possible allele combinations or genotypes corresponding to unidentified contributors, and therefore further reduce the simulation iterations and time consumption.

As shown in FIG. 3B, scenarios 318 may be validated in step 351. Scenarios proposed by the prosecution and/or the defense may or may not be valid given other evidence or indications. And therefore, scenarios may need to be validated before deconvolution analysis is performed. Scenarios need to be coherent and logical. Validation of the scenarios checks for potential conflicts of input data. As an example, in a validation process, scenarios 318 may be checked against parameters 316. If the number of contributors included in parameters 316 is only two but scenarios 318 have four known contributors, there is a conflict between the input data. When there is a conflict, the validation process may flag the conflict and/or provide the conflict information to the user. The validation process can also be re-performed after the conflict is resolved.

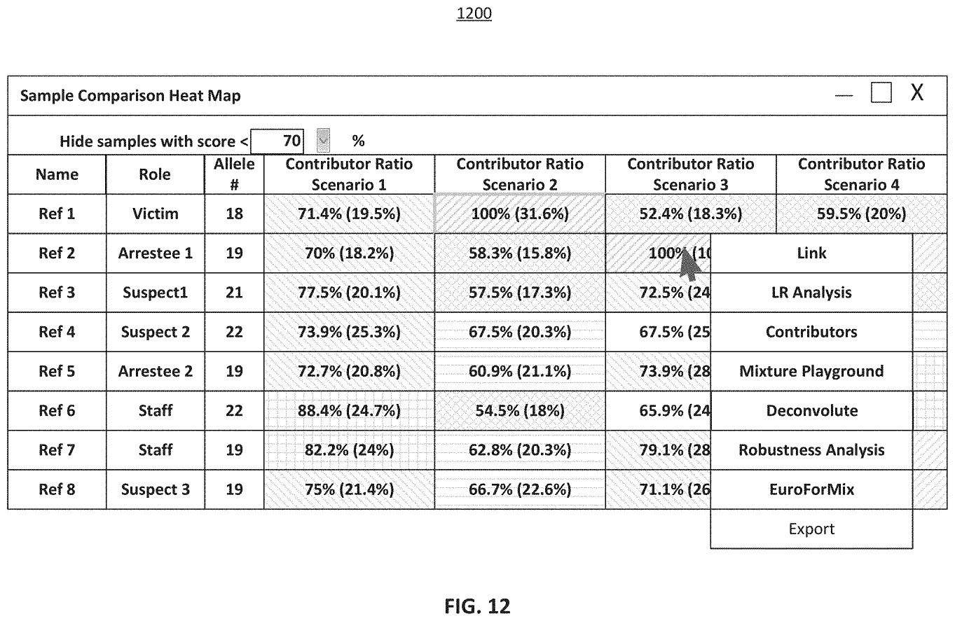

After the scenarios 318 are validated, one or more of evidence profile 312, models 314, parameters 316, and scenarios 318 are used for computing deconvolution in step 352. The deconvolution analysis generates solutions 355 and at least some of the deconvolution solutions can be provided to the user and displayed visually in step 354. FIG. 12 illustrates such a visualization using a heat map. The deconvolution analysis performed in step 352 are described in greater detail below, along with the solutions 355 and visualization step 354.

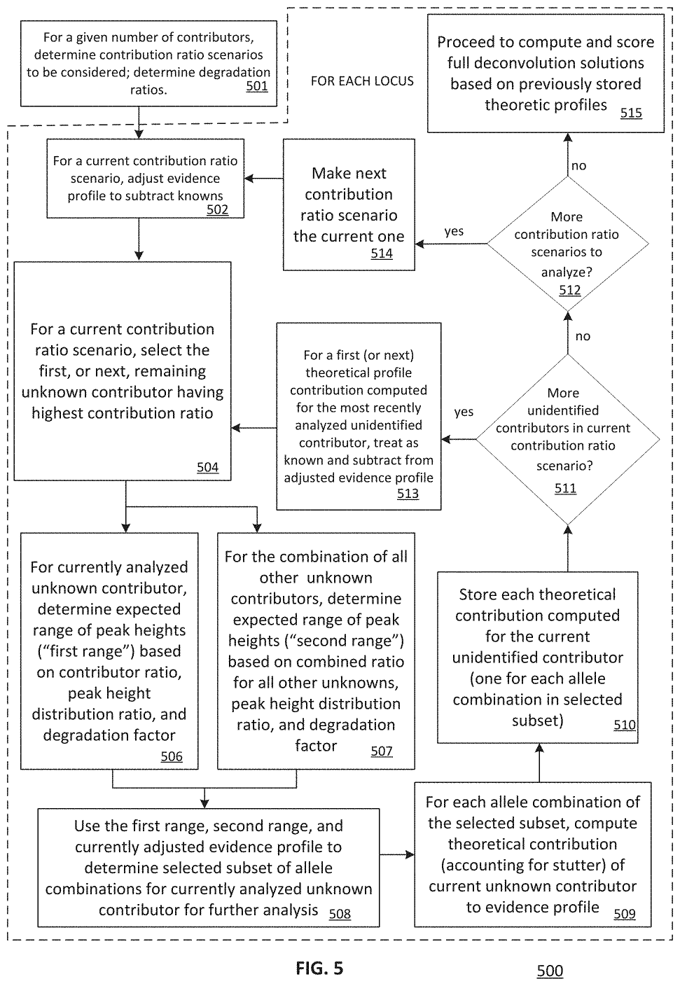

FIG. 5 is a flowchart illustrating an example deconvolution method 500. Method 500 is a computer-implemented method and can be used for implementing step 352 (computing deconvolution) of method 350 shown in FIG. 3B. In some embodiments, method 500 begins with a step 501, in which for a given number of contributors, contribution ratio scenarios to-be-considered are determined. As described above, hypotheses of the number of contributors and their contribution ratios are provided (e.g., by prosecution and defense) as scenarios for consideration. Contribution ratio scenarios can thus be determined from the data provided. In other cases, contribution ratio scenarios can be computed as described below.

In some embodiments, contribution ratio scenarios may or may not be easily ascertained and provided. For example, if there are multiple unidentified contributors and there is a high degree of uncertainty of the amount of contributions from one or more of the unidentified contributors, it may be desirable to account for additional and/or different contribution ratio scenarios in the deconvolution analysis. In some embodiments, contribution ratio scenarios are determined based on a user-configurable ratio resolution step. As an example, if there are two contributors and if the ratio resolution step is configured to be 10%, the possible contribution ratio scenarios comprise 10%-90%, 20%-80%, 30%-70%, 40%-60%, and 50%-50%. As another example, if there are three contributors and if the ratio resolution step is set to be 10%, the possible contribution ratio scenarios comprise (for simplicity, using “1” to represent “10%”, “2” to represent 20%, and so on): 1-1-8, 1-2-7, 1-3-6, 1-4-5, 2-2-6, 2-3-5, 2-4-4, and 3-3-4. Similarly, if there are four contributors and if the ratio resolution step is set to be 10%, the possible contribution ratio scenarios comprise 1-1-1-7, 1-1-2-6, 1-1-3-5, 1-1-4-4, 1-2-2-5, 1-2-3-4, 2-2-2-4, and 2-2-3-3. And if there are five contributors and if the ratio resolution step is set to be 10%, the possible contribution ratio scenarios comprise 1-1-1-1-6, 1-1-1-2-5, 1-1-1-3-4, 1-1-2-2-4, 1-1-2-3-3, 1-2-2-2-3, and 2-2-2-2-2. It is understood that the above contribution ratio scenarios are just examples and other scenarios may be possible (e.g., by changing the ratio resolution step to 5% or 15%).

Step 501 of method 500 also determines sample degradation ratios or values. Sample degradation ratios or values are used in the deconvolution analysis as a factor to account for the fact that the collected biological sample may be degraded. The sample degradation can be caused by, for example, exposing the sample to the sunlight, storing the sample for a long time, sample evaporation, sample drying, etc. In the deconvolution analysis, the sample degradation ratios or values are used to scale the expected or computed peak heights. In some embodiments, the sample degradation ratios or values are configurable and used optionally.

With reference to FIG. 5, after determining the contribution ratio scenarios and the sample degradation ratios, the deconvolution analysis is performed with respect to each locus of a plurality of loci under consideration (e.g., the 20 loci described above, or user-selected ones). In step 502, an adjusted evidence profile is computed by subtracting from the evidence profile a computed expected contribution of all known contributors, if any. For a known contributor, his or her DNA profile can be determined by, for example, analyzing a biological sample from that known contributor. The peak heights in the DNA profile of the known contributor are then be scaled according to the currently analyzed contribution ratio scenario (e.g., scaled according to the percentage of the known contributor with respect to other contributors). The scaled DNA profile of the known contributor can be used as a computed profile of the known contributor, which represents an expected contribution from the known contributor. In some embodiments, stutter peaks are added to the computed profile of the known contributor. As described above, stutter peaks can be computed based on a stutter model empirically or statistically constructed. This process of computing a profile of a known contributor can be repeated multiple times to compute profiles of all known contributors, which represent expected contributions of all known contributors.

The computed profiles of the known contributors are then subtracted from the evidence profile to obtain an adjusted evidence profile. The adjusted evidence profile thus includes only profiles of unidentified contributors. The adjusted evidence profile is then used for further deconvolution analysis. In some embodiments, full peak heights of the computed profiles of the known contributors are subtracted from the evidence profile. In some embodiments, due to peak height imbalance (PHI), subtraction of the computed profiles of the known contributors from the evidence profile uses the lower bound of a range of an expected peak height instead of a full peak height. That is, the lower bound of the range of the expected peak height in the computed profile is subtracted from the evidence profile.

Peak height imbalances are usually computed in heterozygous loci (containing two alleles from different parents). A peak height imbalance may occur when there is greater than a certain percentage of (e.g., 30%) difference in the heights of the two peaks of the two alleles in a heterozygous locus. Typically, a sample from a single person should contain peaks that are roughly the same in peak height. Therefore, if there is a peak height imbalance, it may indicate that the sample is a mixture. However, peak height imbalance can also be caused by other factors such as variability in amplification, pipetting, and electrokinetic injection. In subtracting a computed profile of a known contributor, if the lower bound of the range of expected peak height in the computed profile is subtracted from the evidence profile, the remaining peaks in the evidence profile may on average have higher peak heights than if the full peak heights in the computed profile are subtracted. Therefore, in some embodiments, further deconvolution analysis may account for this situation by using a proportion factor to scale the upper bound of the range of expected peak heights higher. For example, as described in greater detail below, peak height imbalance is used to determine expected ranges of peak heights for selecting subset of allele combinations for further analysis. The upper bound of the expected range of peak heights used for such selection may be adjusted by subtracting the computed profile from the evidence profile (e.g., subtracting the full peak heights, or subtracting the lower bound of the expected range of peak heights).

Referencing FIG. 5, for a current contribution ratio scenario, step 504 of method 500 selects a highest remaining unidentified contributor corresponding to the adjusted evidence profile. For example, there may be total of three contributors including two unidentified contributors and one known contributor. The contribution ratio of the three contributors may be 10%-30%-60% with 10% being the percentage of the known contributor's contribution. In this contribution ratio scenario, the unidentified contributor having the 60% contribution is selected for analysis first and the unidentified contributor having the 30% contribution will be selected for analysis in the next iteration.

Once the highest remaining unidentified contributor is selected, a first range of expected peak heights of this unidentified contributor and a second range of expected peak heights of all other remaining unidentified contributors are computed.

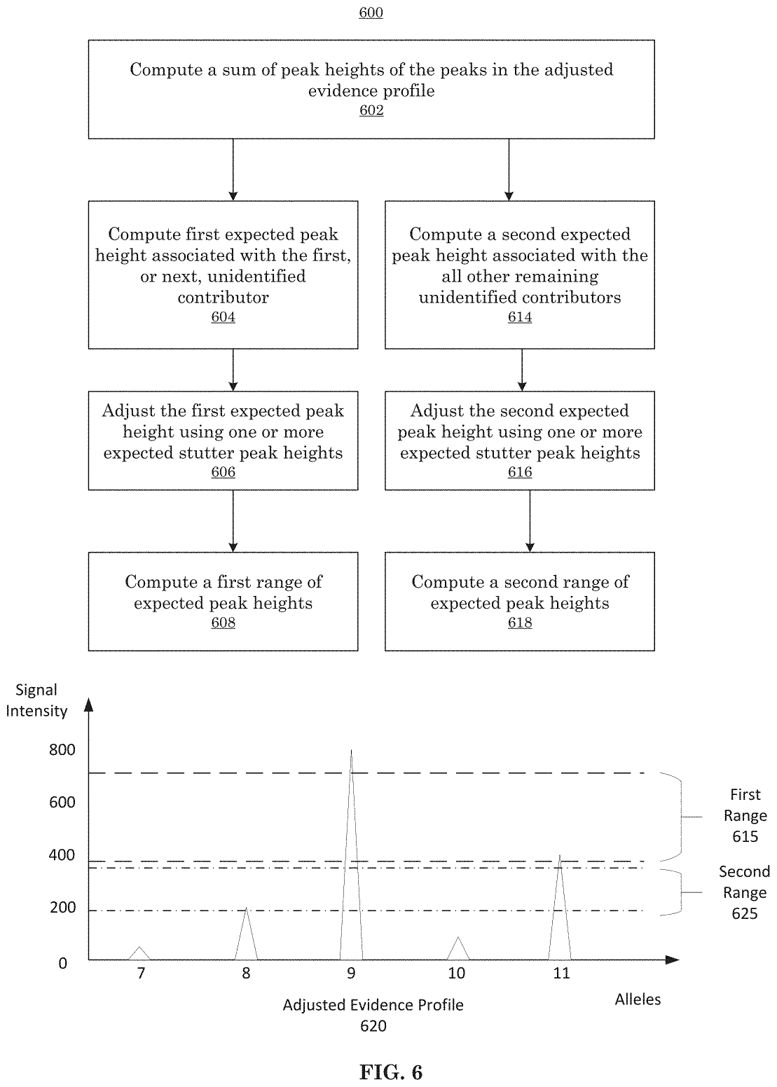

FIG. 6 illustrates a method 600 of computing a first range 615 and a second range 625. The first range of the expected peak heights represents a range of expected peak heights, accounting for peak height imbalance of the highest remaining unidentified contributor. The second range of the expected peak heights represents a range of expected peak heights, accounting for peak height imbalance, of all other remaining unidentified contributors combined.

Method 600 can be used to implement steps 506 and 507 of method 500 in FIG. 5. Step 602 of method 600 computes a sum of peak heights in the adjusted evidence profile. Using adjusted evidence profile 620 in FIG. 6 as an example, there are total of 5 peaks with peak heights of about 50, 200, 800, 80, and 400 for alleles 7, 8, 9, 10, and 11, respectively. The sum of these peaks is therefore about 1530.

Step 604 computes an expected allele peak height that would result from the currently analyzed contributor (i.e., the first or the highest remaining unidentified contributor in the current iteration) having one of the alleles. The computation is based on the sum of the peak heights (from the adjusted evidence profile), the currently analyzed contribution ratio scenario, and a selected degradation value. For example, in a currently analyzed scenario that assumes two unidentified contributors and an 80%-20% contribution ratio, the sum of the expected peak heights of the major contributor is about 0.8*1530=1224. This sum of expected peak heights is for two alleles because a person's DNA profile has two alleles, one from each of the person's parents. Therefore, for one allele of this major contributor, the expected peak height is about 1224*0.5=612. In some embodiments, a degradation value is used to scale down the contribution ratios. E.g., a 0.8 contribution might be scaled down to 0.6 or some other value lower than 0.8 using a degradation function and parameter.

In a similar fashion, step 614 computes the expected allele peak height that would result from all of the other unidentified contributors having one of the alleles. In the above example, there is only one other unidentified contributor with a 20% contribution. For that contributor having two of the same allele, the expected height would be 0.2*1530=306. For one allele of this minor contributor, the expected peak height is about 306*0.5=153. The result is the same if there are two such “minor” unidentified contributors, each with a 10% contribution. Again, accounting for sample degradation, the contribution factor of 0.2 in this example would be scaled to a value lower than 0.2.

Steps 606 and 616 adjust the first and second expected peak heights computed above by removing estimated stutter peaks. Stutter peaks are small peaks that occur immediately before or after a real peak. During the PCR amplification process, the polymerase can lose its place when copying a strand of DNA, e.g., slipping forwards or backwards for a number of base pairs. This may result in stutter peaks. Therefore, in steps 606 and 616, the expected peak heights computed above are adjusted to account for the stutter peak heights. Continuing the above example, using a stutter model, the expected peak heights can be adjusted, for example, from about 612 to about 550 for the major contributor and from about 153 to about 137 for the minor contributor.

Steps 608 and 618 use the first and second expected peak heights and a peak height imbalance (PHI) factor to compute a first range 615 of expected peak heights (for the currently analyzed unidentified contributor, also sometimes referred to herein as the “major” unidentified contributor) and a second range 625 of expected peak heights (for all other unidentified contributors combined, sometimes referred to herein as the “minor” contributor or contributors).

As described above, the peak height imbalance ratio distribution can be obtained from an empirical or statistical model. In an ideal situation, peaks of the same contributor would have roughly the same peak height. But due to stochastic effects, the peaks are not perfectly the same height. Stochastic effects are effects that occur by chance due to various factors and variables associated with analyzing a biological sample to obtain the evidence profile. Peak height imbalance (PHI) or variation due to stochastic effects can usually be modeled using a Gamma distribution. A Gamma distribution can be defined by its shape, scale, and threshold parameters. In some embodiments, the threshold parameters for the Gamma distribution used to represent peak height imbalance or variation are set to about 0.4-0.6.

In the example shown in FIG. 6, a peak height imbalance value of 0.6 is selected using the Gamma distribution and used for computing the first range 615 and second range 625. Using a PHI value of about 0.6 and the adjusted expected peak height of the major contributor, the variation of the adjusted expected peak height of the major contributor can be computed to be about 0.6*550=330. Thus, the first range 615 of the expected peak height can be computed to be between about 385 (i.e., 550−330/2) and about 715 (i.e., 550+330/2).

Using the same PHI value of about 0.6 and the adjusted expected peak height of all the minor unidentified contributors (in this case, one minor contributor), the variation of the adjusted expected peak height of the minor contributor can be computed to be about 0.6*274≈164. Thus, the second range 625 of the expected peak height can be computed to be between about 192 (i.e., 274−164/2) and about 356 (i.e., 274+164/2).

In the above example, two unidentified contributors (i.e., the major contributor having 80% contribution and the minor contributor having 20% contribution) are used for illustration purposes. It is understood that more unidentified contributors may be possible and the computing of the first range and second range can be carried out similarly. For example, if there are total of 3, 4, or 5 unidentified contributors, the first range can still be computed in a similar way as described above using the contribution ratio of the currently analyzed unidentified contributor (e.g., the first or the highest remaining unidentified contributor). The second range can be computed in a similar way as described above using the contribution ratios of all other remaining unidentified contributors, instead of just the one minor contributor as in the above example.

In the above example shown in FIG. 6, the first range 615 is above the second range 625 and the two ranges do not overlap. However, depending on the various scenarios of the total peak heights, contributor ratios, peak height imbalances distributions, degradation ratios, etc., the first range and second range may have other relations. For example, the first range and the second range may be further apart or may overlap with each other. In other examples, the second range may be above the first range.

The first and second ranges are used in step 508 of method 500 of FIG. 5 to select which genotypes correspond, or possibly correspond, to the currently analyzed unidentified contributor (e.g., the first or the highest remaining unidentified contributor). Importantly, the selected genotypes comprise fewer genotypes than a total number of genotypes potentially associated with a current locus in the general population.

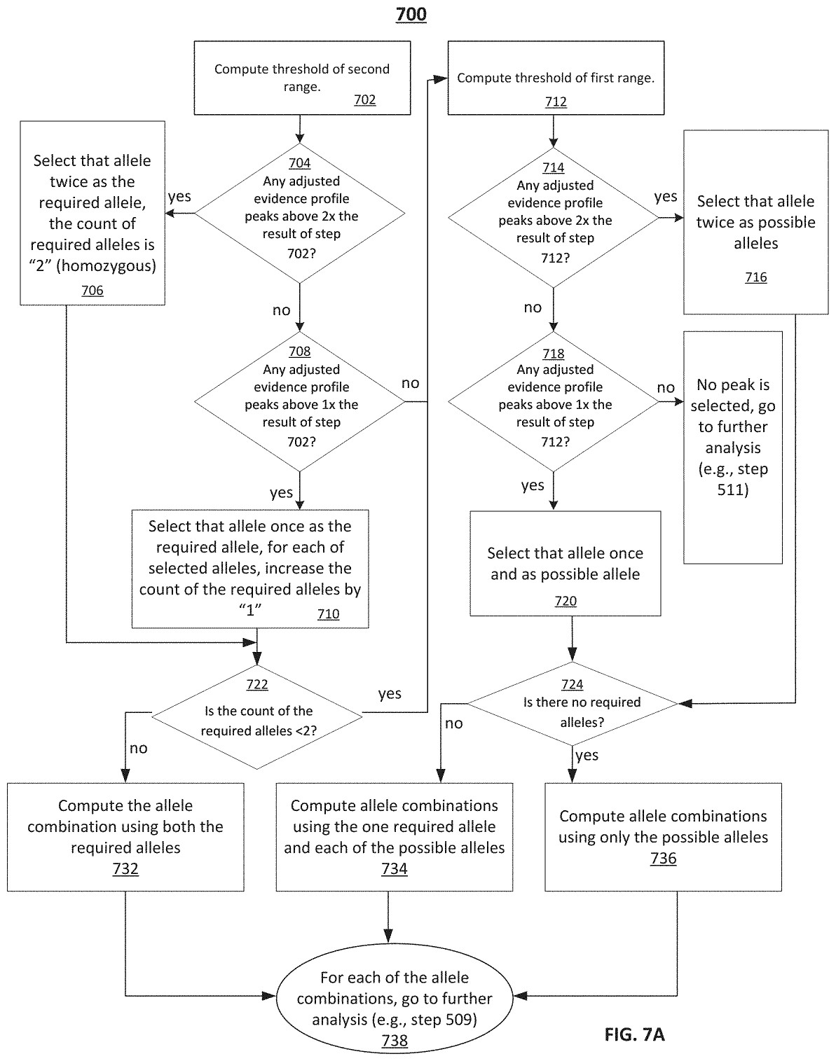

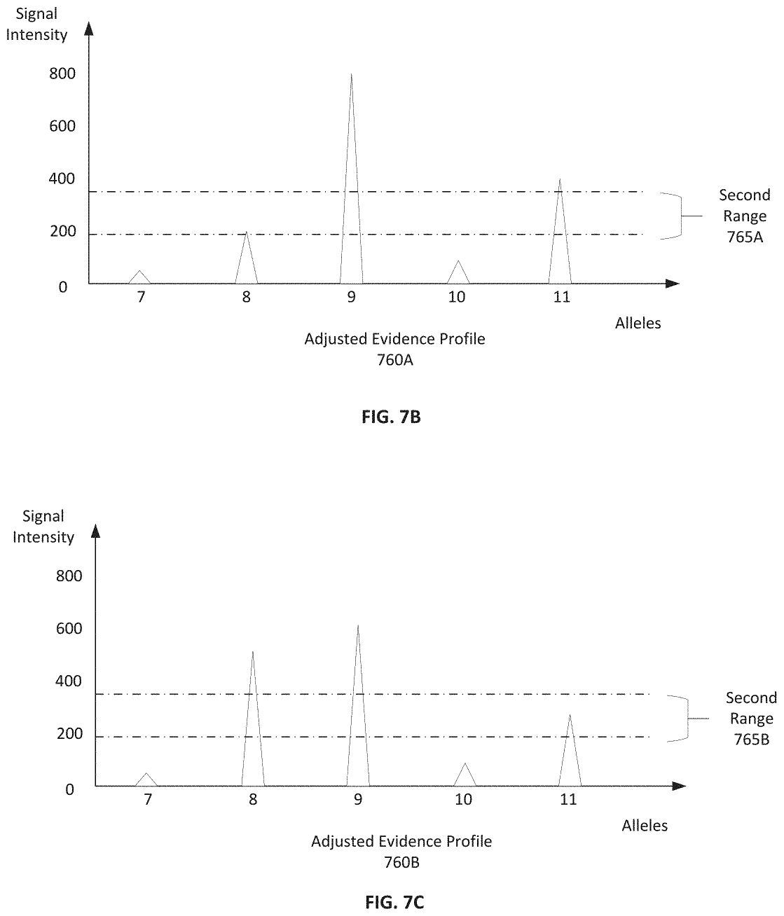

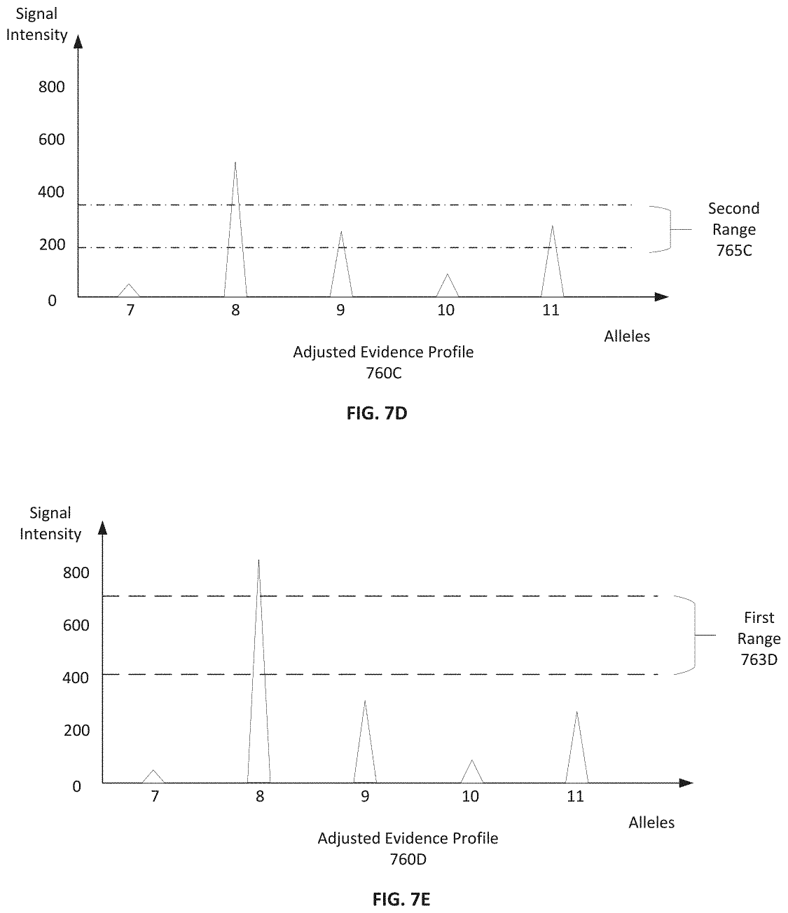

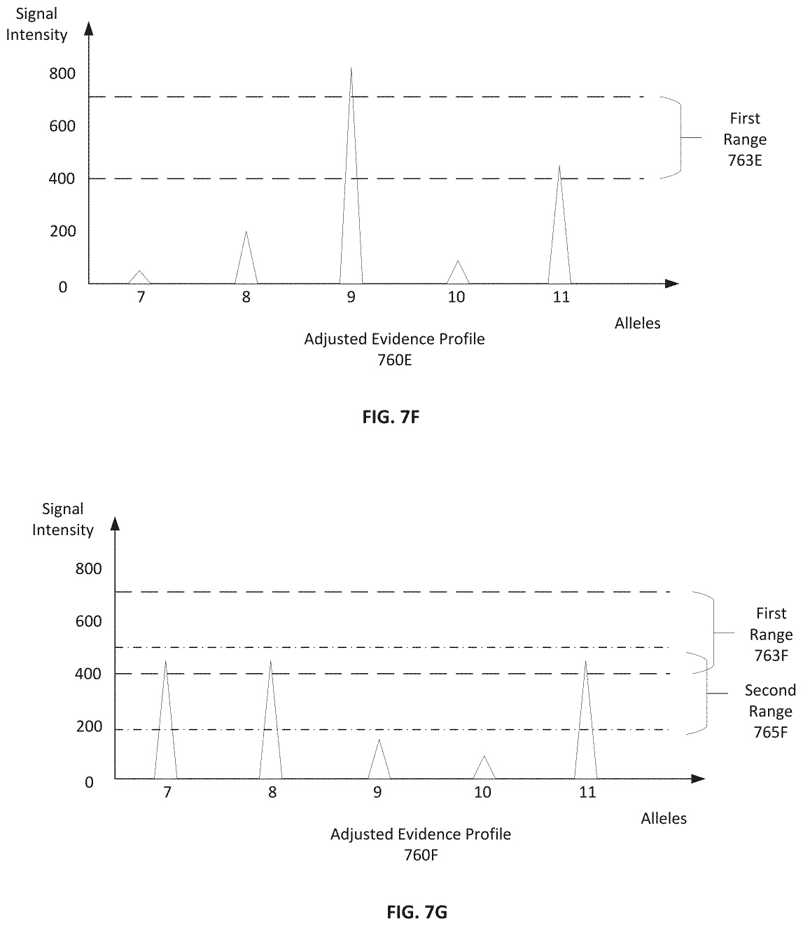

FIG. 7A illustrates method 700 for implementing step 508 of method 500 in FIG. 5. FIGS. 7B-7G show different examples of adjusted evidence profiles to which method 700 might be applied.

Referring to FIG. 7A, step 702 computes a threshold of the second range. The threshold of the second range is the value of the upper bound of the second range. As an example, if the upper bound of second range 765A is about 360 as shown in FIG. 7B, a threshold of second range 765A can thus be configured or selected to be about 360. Step 704 determines whether any peaks in the adjusted evidence profile have a peak height that is at least double the threshold of the second range.

FIG. 7B illustrates such a scenario. In adjusted evidence profile 760A shown in FIG. 7B, allele 9 has a peak with a peak height of about 800, which is more than double 360, the threshold of the second range 765A. Thus, if the result of step 704 is yes (as it is for the example shown in FIG. 7B), step 706 selects allele 9 twice as a pair (9 9), which is the only combination selected for further analysis. In this example, the peak height of allele 9 is so high that the peak can only be explained by the currently analyzed contributor being homozygous for the allele. As a result, both alleles (e.g., in this case 9 and 9) are required to be selected. In this case, both alleles for the currently analyzed unidentified contributor have been found. The count of required alleles is thus increased by 2.

Step 722 checks to see if the count of the required alleles is less than 2. In the above case, both alleles have been found and thus the count is 2. The answer to the determination in step 722 is thus “no”. Accordingly, method 700 proceeds to step 732, where the only allele combination is computed using the same allele twice. After that, the process goes to further analysis (e.g., step 509 in FIG. 5) for this allele combination.

In some embodiments, using the adjusted evidence profile and one or more of the first range and the second range, it is determined if an allele in the adjusted evidence profile has a peak that is at least above the threshold, but below the double of the threshold, of the second range. If so, that allele is selected as a required allele for one of the two alleles for a selected genotype. For example, referencing FIGS. 7A and 7C, if the determination in step 704 is “no” (i.e., there is no peak that has a height that is at least double the threshold of the second range), then method 700 proceeds to step 708. Step 708 determines whether there are any adjusted evidence profile peaks that are above the threshold of the second range. If so, step 710 selects the allele once as a required allele.

FIG. 7C illustrates one such scenario. In FIG. 7C, the threshold of second range 765B is assumed to be about 360 (i.e., the upper bound of second range 765B). In adjusted evidence profile 760B shown in FIG. 7C, for example, two peaks for alleles 8 and 9 respectively have peak heights that are above the threshold of second range 765B (e.g., above the upper bound of second range 765B). As a result, these two peaks are each selected once. In other words, the currently analyzed contributor is determined to be heterozygous, having both allele 8 and allele 9.

In this scenario, for each of the selections of the two alleles, the count of the required alleles is increased by “1”. The total count is thus increased by 2. This allele pair is thus the only combination selected for further analysis. This is because these two peaks can only be explained as heterozygous for the allele for this unidentified contributor. As a result, both alleles (e.g., in this case 8 and 9) are required to be selected. In this case, both alleles for the currently analyzed unidentified contributor have been found. Method 700 of FIG. 7A proceeds to step 722 to check if the count of the required alleles is less than 2. In this scenario, the answer to the determination in step 722 is also “no”. Therefore, the method 700 proceeds to step 732, where the only allele combination is computed using the alleles 8 and 9. After that, the process goes to further analysis (e.g., step 509 in FIG. 5) for this allele combination.

FIG. 7D illustrates another scenario where only one peak, instead of two peaks, is determined to be above the threshold of second range 765C. In FIG. 7D, the threshold of second range 765C is assumed again to be about 360 (i.e., the upper bound of second range 765C). In adjusted evidence profile 760C shown in FIG. 7D, for example, only the peak of allele 8 has a peak height that is above the threshold of second range 765C. The peak of allele 9 is below the threshold of second range 765C. As a result, the peak of allele 8 is selected once. The peak of allele 9 is not selected. In this case, the allele 8 may form a heterozygous allele pair with one of other possible alleles. The count of the required alleles is thus increased by 1. Allele 8 is the only allele that is required to be selected. Therefore, only one allele for the currently analyzed unidentified contributor has been found.

Referencing back to FIG. 7A, step 722 of method 700 checks if the count of the required alleles is less than 2. In the above scenario where only one allele is found, the answer to the determination in step 722 is “yes”. Method 700 thus proceeds to step 712 and other following steps to select other possible alleles, as described in detail below. After selecting one or more possible alleles as described below, step 734 of method 700 computes the allele combinations using the this required allele (e.g., allele 8) and all other possible alleles. After that, the process goes to further analysis (e.g., step 509 in FIG. 5) for this allele combination.

As described above, step 704 of method 700 determines if an allele in the adjusted evidence profile has a peak that is high enough above (e.g., at least double) the threshold of the second range that an analyzed unidentified contributor should be treated as homozygous for the allele. Step 708 determines if any two alleles in the adjusted evidence profile have corresponding peaks that are above the threshold of the second range (but not double). If any of these two determinations is “yes”, a pair of alleles for the currently analyzed unidentified contributor comprise only required alleles. These two required alleles are then both selected and no further selection is needed. The process can proceed to perform further analysis and then to the next iteration of analyzing the next unidentified contributor.

Referencing FIG. 7A, if the count of the required alleles is fewer than 2, method 700 proceeds to steps 712 and 714 to determine if an allele in the adjusted evidence profile has a peak that is at least double a threshold of the first range. If so, that allele is selected twice as possible alleles for each of the one or more selected genotypes. For example, if the determination in step 708 is “no” or that the determination in step 722 is “yes”, method 700 proceeds to step 712 to compute the threshold of the first range. The threshold of the first range is the value of the lower bound of the first range. As an example, if the lower bound of first range 763D is about 400 as shown in FIG. 7E, a threshold of first range 763D can thus be configured or computed to be about 400. Using the threshold of the first range, step 714 of method 700 determines if there are any adjusted evidence profile peaks having peak heights that are at least double the threshold of the first range. If so, step 716 selects that allele twice as two possible alleles.

FIG. 7E illustrates such a situation. In adjusted evidence profile 760D, as shown in FIG. 7E, the peak of allele 8 has a peak height that is about 850, which is more than twice of the lower bound of first range 763D (e.g., about 400). As a result, the peak of allele 8 is selected twice as possible alleles, because this allele may form a possible homozygous allele pair when it is selected twice (e.g., allele pair (8 8)). In this case, two possible alleles for the currently analyzed unidentified contributor have been found (e.g., alleles 8 8). This allele can also form one or more possible allele pair combinations with other possible alleles (e.g., combination (8, 9), (8, 11), etc.).

Referencing FIG. 7A, after selecting the allele twice as possible alleles in step 716, method 700 proceeds to step 724 to check if there are no required alleles. If the answer in step 724 is “no” (e.g., there is currently one required allele found), method 700 proceeds to step 734 to compute all allele combinations using the one required allele and all possible alleles. But if there is no required allele found (i.e., the answer in step 724 is “yes”), method 700 proceeds to step 736 to compute allele combinations using only the possible alleles. In step 738, each of these allele combinations are added to the selected subset for further deconvolution analysis. The process described above is then repeated multiple times until there is no remaining unidentified contributor.

Referencing FIG. 7A, if there are no peaks in the adjusted evidence profile above double the first range (i.e., answer to step 714 is “no”), method 700 proceeds to step 718 to determine if an allele in the adjusted evidence profile has a peak that is above the threshold of the first range. If so, step 720 selects the allele as at least one of two possible alleles for each of the selected genotypes.

FIG. 7F illustrates such a scenario. In adjusted evidence profile 760E shown in FIG. 7F, the threshold of first range 763E is assumed to be its lower bound of about 400. A peak of allele 11 has a peak height that is above the threshold but below double the threshold. The peak of allele 9 would be above double the threshold and should already have been selected in the previous step (e.g., step 714). As a result, the peak of allele 11 is selected once as a possible allele for the currently analyzed unidentified contributor, because this allele 11 may form a heterozygous allele pair with another allele. In this case, one possible allele for the currently analyzed unidentified contributor has been found (e.g., allele 11).

After step 720, method 700 proceeds to step 724 to check if there are no required alleles. If the answer in step 724 is “no” (i.e., there is a required allele), method 700 proceeds to step 734 to compute all allele combinations using the one required allele and all possible alleles. If there are no required alleles (i.e., the answer in step 724 is “yes”), method 700 proceeds to step 736 to compute allele combinations using only the possible alleles. In step 738, each of these allele combinations (e.g., allele combinations having required allele(s) or having only possible allele(s)) are added to the selected subset for further deconvolution analysis. In other words, for each of these allele combinations, the deconvolution analysis goes to the next iteration for analyzing the next unidentified contributor. In step 718, if the answer is “no”, that means there is no peak to be selected. The method 700 the proceeds to the next iteration (e.g., goes to step 511 to see if there are any more unidentified contributors to be analyzed).

As described above, step 714 determines if an allele in the adjusted evidence profile has a peak that is at least double the threshold of the first range. Step 718 determines if an allele in the adjusted evidence profile has a peak that is above, but not double, the threshold of the first range. If any of the two determinations is “yes”, at least one allele is selected for the currently analyzed unidentified contributor because it is a possible allele for explaining a peak in the profile of the currently unidentified contributor. Using the selected possible alleles and a required allele, if any, method 700 computes allele combinations in step 734 or step 736. For each of allele combinations, the analysis of the next unidentified contributor is carried out. As an example, if allele 11 is a required allele and alleles 5, 6, 7 are possible alleles (but not required), allele 11 is selected for this currently analyzed unidentified contributor. For each of the possible alleles 5, 6, and 7, step 734 computes three possible allele combinations, i.e., (11, 5), (11 6), and (11 7), for the currently analyzed unidentified contributor. For each of these allele combinations, the analysis of the next unidentified contributor is carried out. The process described above is then repeated multiple times until there is no remaining unidentified contributor.

FIG. 7G illustrates another example wherein only possible alleles are found. In adjusted evidence profile 760F shown in FIG. 7G, for example, the peaks of alleles 7, 8, and 11 have peak heights above the threshold of the first range 763F (e.g., the lower bound of first range 763F) but are not double the threshold of the first range 763. As a result, these peaks for alleles 7, 8, and 11 are selected as alleles to form possible allele combinations for the currently analyzed unidentified contributor, because these alleles may form heterozygous allele pairs with one another (e.g., (7 8), (7 11), and (8 11)). In this case, only possible alleles for the currently analyzed unidentified contributor are found (i.e., alleles 7, 8, and 11), but no required allele is found. Because there are no required alleles, the answer to the determination in step 724 (i.e., whether there is no required alleles) is “yes”. Therefore, method 700 proceeds to step 736 to compute all allele combinations using only the possible alleles. In step 738, each of these possible allele combinations is added to the selected subset for further deconvolution analysis. In other words, for each of these allele combinations, the deconvolution analysis goes to the next iteration for analyzing the next unidentified contributor.

FIG. 7A illustrates one example method 700 of selecting subsets of allele combinations for the currently analyzed unidentified contributor for further analysis. It is understood that additional steps may be added to method 700, existing steps may be removed from method 700, and/or the order of the steps may be changed. For example, steps 704 and 708 may be performed in parallel, so do steps 714 and 718.

Using the above selection method illustrated in FIG. 7A, fewer genotypes are selected for further deconvolution analysis than a total number of genotypes potentially associated with a locus in a general population. As a result, the deconvolution analysis only needs to be performed with respect to scenarios of allele combinations that can at least potentially be used to explain the mixed DNA evidence profile of the one or more the unidentified contributors. In other words, the selection method described above eliminates many allele combinations that are random or impossible for explaining the mixed DNA evidence profile. Thus, the full deconvolution analysis is much more efficient and can provide analysis result much faster.

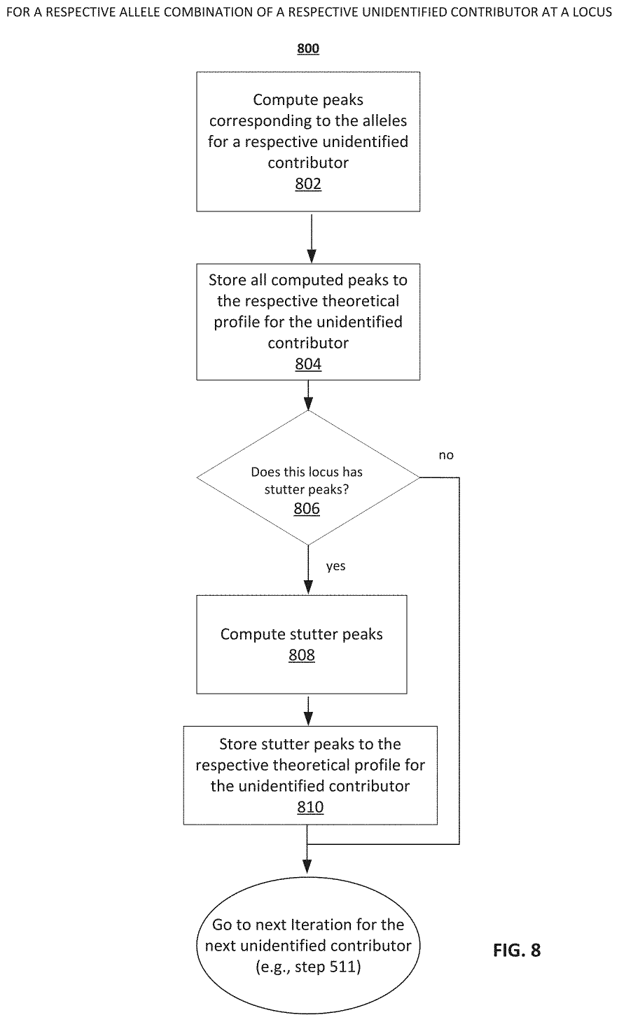

Referencing back to FIG. 5, for each of the allele combinations of the selected subset genotypes, step 509 of method 500 computes theoretical contributions from each of the allele combinations. Step 510 stores the theoretical contribution for computing a theoretical profile. FIG. 8 illustrates an example method 800 for computing theoretical contributions. Method 800 can be used to implement steps 509 and 510 in method 500 of FIG. 5.

Method 800 can be performed for each of the allele combinations of a respective unidentified contributor at a locus. For example, for each unidentified contributor, one or more allele combinations may be selected (e.g., (5 5), (5 6), (5 7), etc.). Then for each of these allele combinations for a particular unidentified contributor, method 800 can be performed to compute theoretical contributions of the alleles. The process then goes to the next iteration shown in FIG. 5 (step 511) to determine the next unidentified contributor and select allele combinations for this next unidentified contributor. And method 800 can be repeated for all the allele combinations to compute their theoretical contributions. As one example, assuming that the first allele combination is (5 5) for the first unidentified contributor, method 800 is performed to compute the theoretical contributions of this allele pair (5 5). Then the process goes to the next iteration for the next unidentified contributor and selects an allele combination of (6 6). Method 800 is then performed to compute theoretical contributions of this allele pair (6 6). Further assuming there are only these two unidentified contributors in this scenario, then the computed theoretical contributions are stored for further computing a theoretical profile, which is described in more detail below.

Referencing FIG. 8, for one allele combination (e.g., 5 5) of a respective unidentified contributor, step 802 computes the peaks corresponding to the alleles in the allele combination. In particular, in step 802 theoretical peak heights are computed using the currently analyzed contribution ratio scenario for the respective unidentified contributor, the sample degradation ratio, and the peak height imbalance (PHI) value. For example, if the sum of the total peak heights in the adjusted evidence profile is 1000 and the contribution ratio for the respective unidentified contributor is 80%, then the theoretical peak heights for a homozygous allele combination (e.g., 5 5) are computed to be 400. The theoretical peak heights can be scaled or adjusted by the sample degradation ratio and/or the peak height imbalance value. Using the theoretical peak heights, expected peaks are computed.

Step 804 stores the computed peaks for computing a respective theoretical profile or adds the computed peaks directly to the respective theoretical profile. Step 806 determines if the currently analyzed locus has stutter peaks. This determination can be performed using, for example, a stutter model for the currently analyzed locus. If the answer is “yes”, stutter peaks are computed in step 808. For example, depending on the locus, one or more of forward stutter peaks, backward stutter peaks, double backward stutter peaks, half forward stutter peaks, half backward stutter peaks may be computed. Step 810 stores these computed stutter peaks for computing a respective theoretical profile or adds them directly to the respective theoretical profile. Method 800 can then go to next iteration for the next unidentified contributor.

Referencing back to FIG. 5, after storing theoretical contributions computed for the current unidentified contributor in step 510, method 500 proceeds to step 511 to determine if there are more unidentified contributors in the current contribution ratio scenario. If so, method 500 proceeds to step 513 for the next unidentified contributor. In step 513, the most recently analyzed unidentified contributor is treated as a known contributor. Next, the theoretical contributions computed for the current unidentified contributor are subtracted from the adjusted evidence profile (just like subtracting the known contributors from the original evidence profile as described above). The further adjusted evidence profile (i.e., with the contributions from the known contributor and the most recently analyzed unidentified contributor subtracted) is used for the next iteration. Method 500 proceeds to repeat the process as described above from step 504.

Referencing still to FIG. 5, if the answer in step 511 is “no” (i.e., there are no more unidentified contributors in the current contribution ratio scenario), method 500 proceeds to step 512 to determine if there are more contribution ratio scenarios. As described above, there may be different combinations of the contribution ratio scenarios even if the number of contributors remains the same. For example, for the same 3 contributors, the contribution ratio scenarios may include 1-1-8, 1-2-7, 1-3-6, 1-4-5, 2-2-6, 2-3-5, 2-4-4, etc. Therefore, each of these contribution ratio scenarios may need to be analyzed.

If step 512 determines that there are more contribution ratio scenarios, method 500 proceeds to step 514, in which the next contribution ratio scenario is treated as the current contribution ratio scenario. The process then repeats from step 502, in which the original evidence profile is adjusted by subtracting the profile of known contributors. The rest of the process is similar to those described above and thus not repeated.

If step 512 determines that there are no more contribution ratio scenarios, method 500 proceeds to step 515. In step 515, the process proceeds to compute theoretical profiles using the previously stored theoretical contributions for all the allele combinations and contribution ratio scenarios. The computed theoretical profiles are compared to the original evidence profile and scored. The processes of computing and scoring the theoretical profiles are described next.

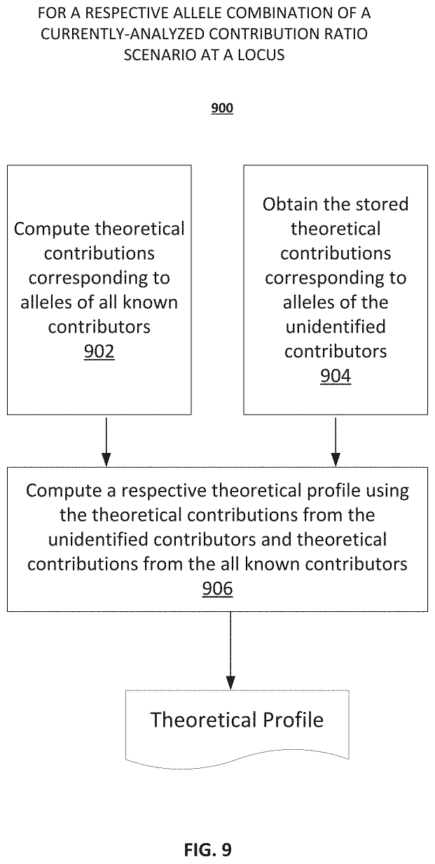

FIG. 9 is a flowchart illustrating an example method 900 for computing a theoretical profile for an allele combination of a currently analyzed contribution ratio at a locus. Method 900 can be repeated for computing theoretical profiles for each allele combination, each contribution ratio scenario, and at each locus. Step 902 of method 900 computes theoretical contributions corresponding to alleles of all known contributors. As described above, for a known contributor, his or her DNA profile can be determined by, for example, analyzing a biological sample from that known contributor. The peak heights in the DNA profile of the known contributor can then be scaled according to the currently analyzed contribution ratio scenario (e.g., scaled according to the percentage of the known contributor with respect to other contributors). The scaled DNA profile of the known contributor is used as a theoretical profile of the known contributor. This theoretical profile represents expected contributions from the known contributor. In some embodiments, theoretical stutter peaks are added to the theoretical profile of the known contributor. As described above, stutter peaks can be computed based on a stutter model empirically or statistically constructed.

Step 904 of method 900 obtains the stored theoretical contributions corresponding to the alleles of the unidentified contributors in the respective allele combination. As described above, these theoretical contributions can be computed using, for example, method 800. These theoretical contributions of the unidentified contributors include computed allele peaks and computed stutter peaks.

Step 906 of method 900 computes the theoretical profile for the respective allele combination, the respective contribution ratio scenario, and the respective locus using the theoretical contributions from the unidentified contributors and theoretical contributions from the known contributors. For example, the theoretical profile can be constructed by merging the peaks (e.g., both allele and stutter peaks) of the unidentified contributors and peaks of the known contributors. Using the theoretical profile, a profile score can be computed. The profile score indicates a degree of matching between the theoretical profile and the evidence profile.

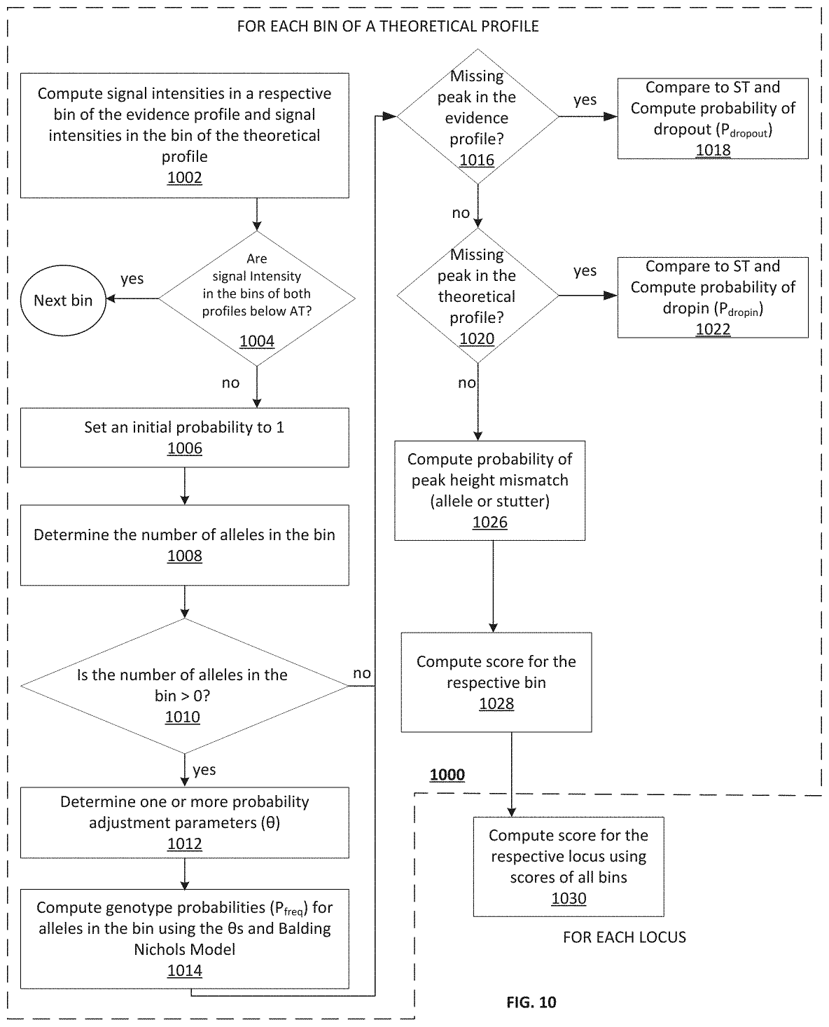

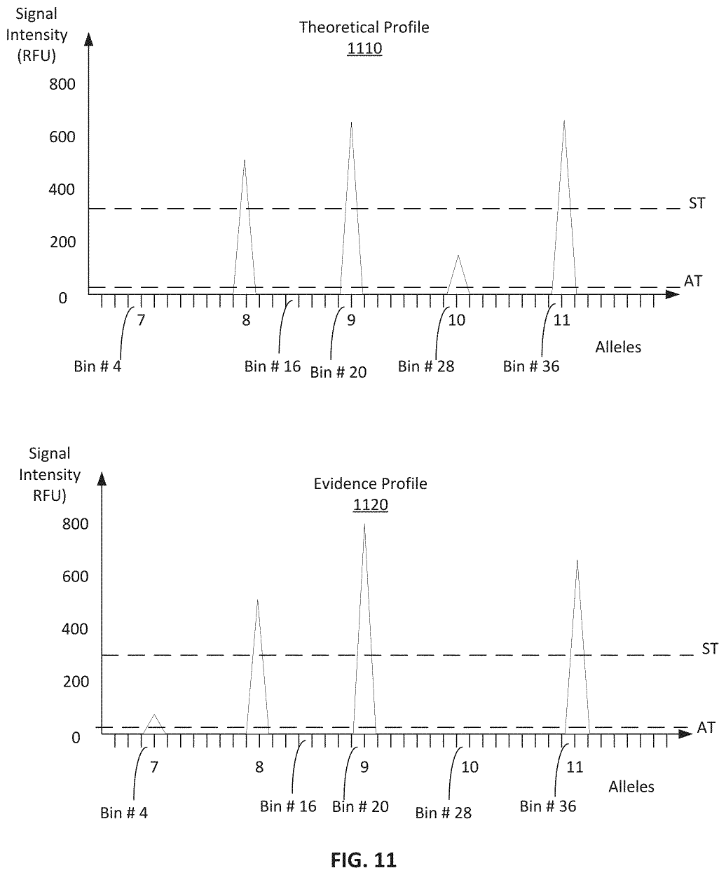

FIG. 10 is a flowchart illustrating an example method 1000 for computing a score of a bin in a theoretical profile. Method 1000 can be repeated to compute a score for each bin and then the profile score can be computed using the scores of the bins. As shown in FIG. 10, step 1002 computes signal intensities in a bin of the evidence profile and signal intensities in the corresponding bin of the theoretical profile. A bin is a measurement interval along the horizontal axis of a profile. The signals in a bin are measured together, e.g., integrated or summed. Referencing FIG. 11 as an example, the horizontal axis of a profile (either a theoretical profile or an evidence profile) represents allele numbers. A bin may be configured or customized to have any desired interval (e.g., smaller than a peak width, equal to a peak width, or larger than a peak width). FIG. 11 illustrates that both the theoretical profile 1110 and the evidence profile 1120 are configured to have about 43 bins (represented by the intervals between the short vertical lines underneath the horizontal axis). Signals in a particular bin may or may not have an allele peak. For example, a bin that is between the peaks of alleles 8 and 9 (e.g., bin #16) in theoretical profile 1110 has only noise and/or only stutter peaks. Regardless of whether the signals in a bin have noise, stutter peaks, and/or allele peaks, the signal intensities in the bin can be computed. In the example illustrated in FIG. 11, the vertical axis represents the signal intensities in RFUs (relative fluorescence units).

Referencing back to FIG. 10, step 1004 determines if signal intensities in a bin of the theoretical profile and the corresponding bin of the evidence profile are below an analytical threshold AT. The analytical threshold AT is typically configured to be above a noise baseline of the analytical instrument. Thus, the analytical threshold AT is a threshold for distinguishing signals versus noise. Examples of the analytical threshold AT are 50 or 75 RFUs. In probabilistic genotyping, a lower value of the analytical threshold AT (e.g., 5 or 10 RFUs) is used to reduce or eliminate the possibility that stutter peaks are undesirably lost. This analytical threshold AT can be user configured or automatically configured based on statistics or models. If the signal intensities in a bin are below the analytical threshold AT, the particular bin probably only includes noises and thus there is no need to compute a score for that bin. The analysis can just move to the next bin. Using bin #16 shown in FIG. 11 as an example, that particular bin of the theoretical profile 1110 and the corresponding bin of the evidence profile 1120 only have noise but no allele peaks or stutter peaks. As such, the computing of signal intensities in bin #16 can be skipped. The process can move to the next bin to compute a score.

If a particular bin has signal intensities that are above the analytical threshold AT in one or both of the evidence profile and the theoretical profile, then the bin probably does not include just noise. Thus, as shown in FIG. 10, method 1000 proceeds to step 1006. Step 1006 sets an initial genotype probability for the bin to 1. The genotype probability (denoted by P_{freq}) represents the frequency of a particular allele. In other words, it represents the probability of seeing the same allele N number of times. A probability of seeing a particular allele (e.g., allele #8) once represents whether the allele is a common allele that is seen in a large population (e.g., 80% of the general population), a rare allele that is only seen in a small population (e.g., 2% of the general population), or anywhere in between. The probability of seeing the same allele N number of times (i.e., P_{freq}) is thus the Nth power of the probability of seeing the allele once. For example, if the probability of seeing an allele once is 0.2 and the allele is selected twice, then P_{freq} is 0.04. The probability of seeing an allele once is also adjusted to account for relation between the contributors (e.g., such a probability increases significantly if a contributor is a sibling of another contributor). The adjustment of the probability of seeing an allele once can be performed using a probability adjustment parameter and is described in more detail below.

Method 1000 next proceeds to step 1008 to determine the number of alleles in the bin. As an example shown in FIG. 11, bin #36 has signals that correspond to an allele peak in both the evidence profile 1120 and the theoretical profile 1110. It is thus determined that the number of alleles in bin #36 of both the evidence profile 1120 and the theoretical profile 1110 is 1. Thus, for bin #36, the total number of alleles is 2 accounting for both the evidence profile 1120 and theoretical profile 1110. As another example, for bin #4, the number of alleles in the theoretical profile 1110 is “0”. But the number of alleles in that bin in the evidence profile 1120 is “1”. As such, bin #4 has a total number of alleles of 1.

Referencing FIG. 10, method 1000 next proceeds to step 1010 to determine if the number of alleles in a bin is greater than 0. Continuing with the above examples of bin #4 and bin #36, the answer in step 1010 is “yes” for both bins. In the example of bin #4, only the evidence profile 1120 has an allele peak (allele 7). There is no allele peak in bin #4 of the theoretical profile 1110.

Referencing still to FIG. 10, if the number of alleles in a particular bin is greater than 0, method 1000 proceeds to step 1012 to determine one or more probability adjustment parameters. Using the probability adjustment parameters and a model (e.g., the Balding and Nichols model), step 1014 of method 1000 computes genotype probabilities (P_{freq}). The Balding and Nichols model is a probability model that takes account of shared ancestry in a population. In this model, the distribution of alleles in the population is assumed to be known. To account for the co-ancestry of individuals, the probability adjustment parameter (denoted by θ) is used. Typically, θ is in the range of 0.01-0.03. By using the probability adjustment parameter θ, the genotype probabilities are perturbed away from those obtained under Hardy-Weinberg equilibrium.

As described above, the genotype probabilities P_{Freq} represents the probability of seeing the same allele N number of times. In general, for a given locus, the allele frequencies in the population can be denoted by the vector P=(p₁, p₂, . . . , pₖ) for the K alleles (A₁, A₂, . . . , Aₖ). The probability of randomly selecting one allele of type Aₖ is P_{Freq}=pₖ. The probability of randomly selecting a second allele of the same type, given that it has already been selected once, is P_{Freq}=θ+(1−θ)pₖ. The probability of randomly selecting a third allele of the same type, given that it has already been selected twice, is P_{Freq}[2θ+(1−θ)pₖ]/(1+θ). In general, the probability of randomly selecting an aₖ-th allele of the same type, given that it has already been selected aₖ−1 times of the same type, is P_{Freq}=[(aₖ−1)θ+(1−θ)pₖ]/[(aₖ−1)θ+1−θ]. By using the Balding and Nichols model, the initial genotype probability is adjusted by θ and an adjusted probability P_{Freq} is computed. In some embodiments, multiple values of θ may be used because different known or unknown contributors may each have a different θ. Thus, different values of θ can be used for each of the contributors. After step 1014 for computing the genotype probabilities, the method 1000 proceed to step 1016.

Referencing with FIG. 10, if in step 1010, the number of alleles in the bin is determined to be 0 (i.e., there is no allele peaks in this bin), method 1000 may skip steps 1012 and 1014 and proceed directly to step 1016. Step 1016 determines if the evidence profile has a missing peak. FIG. 11 illustrates such a scenario using bin #28. By comparing the signal intensities of bin #28 of theoretical profile 1110 and those of bin #28 of evidence profile 1120, it can be determined that the evidence profile 1120 does not have a peak at bin #28. It can also be determined that the theoretical profile 1110 does have a peak at bin #28. In other words, there is a missing peak at bin #28 in the evidence profile 1110.

Referencing FIG. 10, if there is a missing peak in the evidence profile, method 1000 proceeds to step 1018 to compute probability of dropout denoted by P_{dropout}. In some embodiments, before computing the probability of dropout, step 1018 also checks whether a possible dropout peak is above the stochastic threshold ST, and if so, determines that there is no need to compute the probability of dropout or that the probability of dropout can be assumed to be zero. Stochastic threshold ST is often used to account for signals or noises that might occur by chance due to various factors and variables associated with analyzing a biological sample. If a peak is above the stochastic threshold ST, the probability of that peak being caused by noise or being a dropout peak is almost zero. If a peak is below the stochastic threshold ST, then there is a non-zero probability that this peak could be a dropout peak. If step 1018 determines that the probability of dropout needs to be computed, it proceeds to compute the probability of dropout using models or distributions. When computing the probability of dropout, allele drop out or stutter drop out are distinguished. A particular peak may include both contributions from an allele peak and a stutter peak. Therefore, a probability of a dropout for an allele peak and a probability of a dropout for a stutter peak can be separately computed. Computing of the probability of dropout can be performed by applying one or more models, error functions, etc. to the currently analyzed theoretical profile. For example, based on a Gamma distribution modeling the peak height imbalance (PHI) ratios, for an expected height of an allele peak (e.g., about 400 RFU), the probability that the peak height is below the analytical threshold AT (that is, the probability of seeing the peak between zero and AT) can be computed. This probability can be computed by calculating an integral of the probability density between zero and AT to obtain a cumulative probability density. The probability that the peak height is below the analytical threshold AT can be considered as the probability of dropout. For a stutter peak, the probability of dropout can be similarly computed using a distribution for stutter peaks.

If there is no missing peak in the evidence profile, method 1000 proceeds to step 1020 to determine if there is a missing peak in the theoretical profile. FIG. 11 illustrates such a scenario using bin #4. By comparing the signal intensities of bin #4 of theoretical profile 1110 and those of bin #4 of evidence profile 1120, it can be determined that theoretical profile 1110 does not have a peak at bin #4. It can also be determined that evidence profile 1120 has a peak at bin #4. In other words, there is a missing peak at bin #4 in theoretical profile 1110. Or put in the other way, there is an extra peak at bin #4 in evidence profile 1110.

Referencing FIG. 10, if there is a missing peak in the theoretical profile, method 1000 proceeds to step 1022 to compute probability of dropin denoted by P_{dropin}. In some embodiments, before computing the probability of dropin, step 1022 also checks whether a possible dropin peak is above the stochastic threshold ST, and if so, determines that there is no need to compute the probability of dropin or that the probability of dropin can be assumed to be zero. Similar to those described above, if a peak is above the stochastic threshold ST, the probability of that peak being caused by noise or being a dropin peak is almost zero. If a peak is below the stochastic threshold ST, then there is a non-zero probability that this peak could be a dropin peak. If step 1022 determines that the probability of dropin needs to be computed, it proceeds to compute the probability of dropin using models or distributions. A dropin peak may be caused by noise. In some embodiments, computing of the probability of dropin can be performed by applying one or more models, error functions, user-defined distributions, etc. to the currently analyzed theoretical profile. For example, a noise probability can be computed based on a user defined distribution (e.g., a log normal distribution) that depends on the amount of DNA and the locus. The noise probability can be used as the probability of dropin. For example, based on a distribution that models random variables (e.g., the log normal distribution), the probability of seeing a noise peak having a height that is equal to or greater than the height of the dropin peak can be computed by calculating the integral of a probability density from zero to the particular peak height. Therefore, the higher the peak, the less likely the peak is caused by just noise.

If there is no missing peak in both the theoretical profile and the evidence profile at a particular bin, method 1000 proceeds to step 1026 to compute probability of peak height mismatch. FIG. 11 illustrates such a scenario using bin #20. By comparing the signal intensities of bin #20 of theoretical profile 1110 and those of bin #20 of evidence profile 1120, it can be determined that bin #20 of the theoretical profile 1110 has a peak that is smaller than of the corresponding peak at bin #20 of the evidence profile 1120. A peak height mismatch occurs when there is a mismatch of peak heights between the theoretical profile and the evidence profile at the corresponding bin. While FIG. 11 illustrates that at bin #20, the peak height in the theoretical profile 1110 is smaller than the peak height in the evidence profile 1120, it is understood that the other way is also possible. It is also understood that the peak height mismatch can occur for one or both of an allele peak and a stutter peak. A probability of a peak height mismatch can be computed by applying one or more models, error functions, etc. to the currently analyzed theoretical profile. The error function may include, for example, an error squared function (e.g., square the difference of the peak heights and normalize it to 0-1), a percent difference function, and/or a function for computing the ratio of the probabilities based on a distribution (e.g., using the Gamma distribution to compute the ratio of probabilities of the peaks having peak heights in the theoretical profile and in the evidence profile). After the probability of peak height mismatch is computed, method 1000 proceeds to step 1028.

Step 1028 computes a score for the bin using the one or more genotype probabilities (P_{Freq}), the probability of dropout, the probability of dropin, the probability of peak height mismatch, or a combination thereof. For example, if there is a dropout, the score of the bin is equal to the product of a P_{Freq} and P_{dropout}. Similarly, if there is a dropin, the score of the bin is equal to the product of a P_{Freq} and P_{dropin}. If there is no dropout or dropin, then the score of the bin is equal to the product of a P_{Freq} and P_{peak_height_mismatch}.

Method 1000 can be repeated for each bin of a profile to compute a score. Using the scores for all the bins, step 1030 computes a profile score of a theoretical profile for a locus. For example, a profile score may be the product of the scores of all the bins. Then, scores for multiple theoretical profiles, which correspond to multiple scenarios, can be obtained by repeating method 1000 and step 1030 described above. These scores for multiple theoretical profiles can be sorted from high to low, with a higher score indicating a better matching between a theoretical profile and the evidence profile.

In other embodiments, as shown in FIG. 12, a heat map can be used to visualize the scores of multiple theoretical profiles. In a heat map 1210, the scores highlighted in green (e.g., represented by line patterns from top right to left bottom) are high scores (e.g., 100%) indicating a good match between a theoretical profile and the evidence profile. The scores highlighted in yellow (e.g., represented by small square patterns) are medium scores (e.g., 70-79%) indicating a medium match between a theoretical profile and the evidence profile. The scores highlighted in red (e.g., represented by horizontal line pattern or diamond patterns) are low scores (e.g., 50-60%) indicating a relatively low match between a theoretical profile and the evidence profile. Thus, the scores can be displayed using the heat map. It is understood that the heat map can use any number of colors to represent different scores, thereby providing a visual display of the scores for different contributor ratio scenarios. The top scored theoretical profiles may be used to provide likelihood of matching between the unidentified contributors and persons of interest (POIs).

The above-described methods improve the efficiency of genotyping used in, for example, forensic analysis to identify POIs. By intelligently eliminating allele combinations that are random or impossible to result in a good match, the amount of deconvolution analysis is significantly reduced. In turn, the analysis time is reduced as well. The methods thus result in an improved efficiency without sacrificing accuracy.

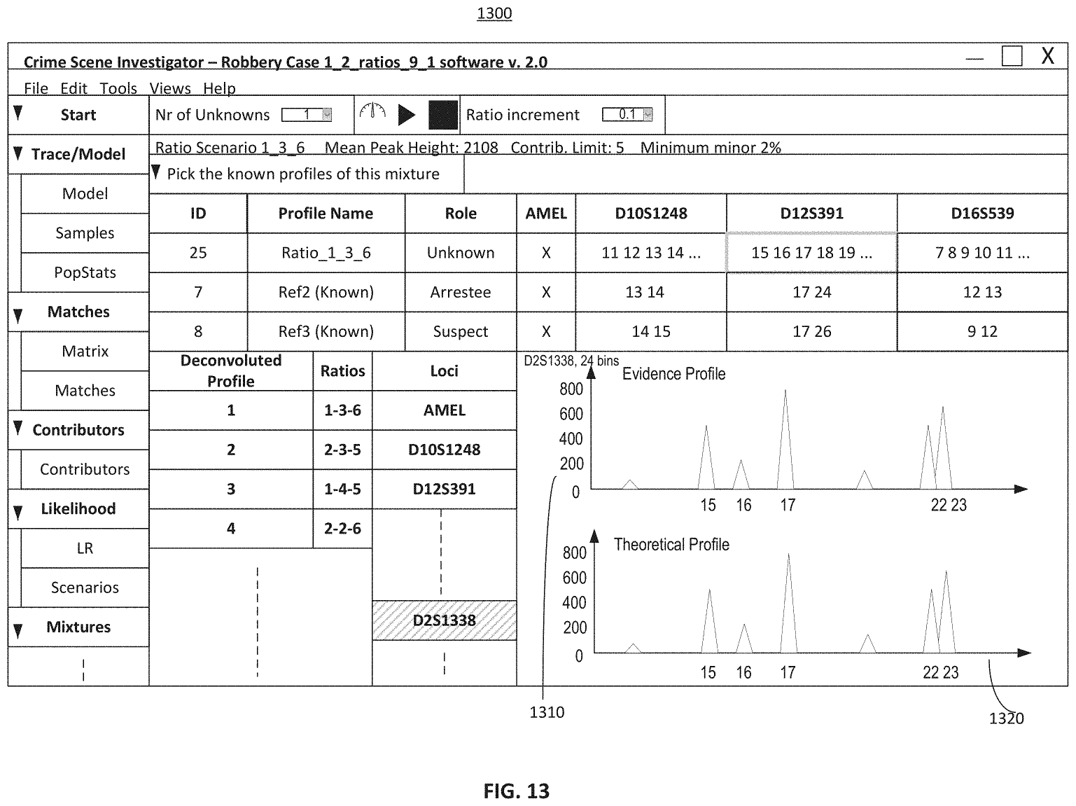

FIG. 13 illustrates an example user interface 1300 used for receiving user inputs and displaying deconvolution analysis results. As shown in FIG. 13, user interface 1300 can include input fields such as number of unknowns, ratio increment, etc. The number of unknowns field allows the user to input the number of unidentified contributors. The ratio increment field allows the user to input the contribution ratio resolution step (e.g., 10%, 5%, etc.). User interface 1300 further provides menu items or selectable fields for viewing detailed information. For example, on the left side of user interface 1300, several menu and sub-menu items are provided. These menu items include trace/model, matches, contributors, likelihood, mixtures, etc. When one of these menu or sub-menu items is selected, user interface 1300 can display relative information. For example, if a “Model” menu is selected, user interface 1300 can display one or more model(s) 314 including distributions related to computing stutter peaks, noise, shoulders, inter locus balance (ILB), peak height imbalance or peak height ratio (PHR), population frequencies and decay, or the like. For example, user interface 1300 can provide detailed information about the stutter model, the PHR model, the ILB model, the noise model, the population statistics, etc. that are used in the deconvolution analysis. As another example, if the “Contributor” menu item is selected, user interface 1300 displays detailed information about the contributors including, for example, their role (victim, suspect, etc.), allele information of each locus, clues, whether known or unknown, etc.

User interface 1300 can also display deconvoluted profile (e.g., the theoretical profiles) together with the evidence profile, as shown in FIG. 13. This way, the user interface 1300 provides a visual and direct comparison between the evidence profile 1310 and the theoretical profile 1320. In some embodiments, user interface 1300 also provides the contribution ratio information and the locus information for a particular theoretical profile. It is understood that FIG. 13 is merely an illustration of a possible user interface 1300. User interface 1300 may include more or fewer icons, menu items, tables, charts, profiles, etc., and may display them in any desired manner.

FIG. 14 illustrates a flowchart of a method 1400 for predicting the number of contributors of a biological sample using an evidence profile obtained from the biological sample. Method 1400 can be used to implement, for example, steps 320 and 330 shown in FIG. 3A. With reference to FIG. 14, in step 1402, the evidence profile, or data representing the evidence profile, is obtained from a system that generates the evidence profile (e.g., the system shown in FIG. 1). As one example illustrated in FIG. 2A, the evidence profile includes data representing peaks at multiple loci. The peaks in the evidence profiles may include allele peaks and/or stutter peaks. Stutter peaks are small peaks that typically occur one repeat unit before or after an allele peak. During the PCR amplification process, the polymerase can lose its place when copying a strand of DNA, e.g., slipping forwards or backwards for a number of base pairs, thereby causing stutter peaks.

Step 1404 of method 1400 receives an indication of a plurality of selected NOC models. As described above, for example, a user interface can be provided for the user to select multiple NOC models. The indication of which NOC models are selected is received and subsequent processes are performed using the selected NOC models. Using a combination of multiple NOC models oftentimes can provide a more accurate prediction of the NOC than using a single model.

Step 1406 of method 1400 obtains weights assigned to each of the selected NOC models. Weights are used to improve the NOC prediction accuracy. For example, if a first NOC model tends to provide more accurate results in general or under certain circumstances than a second NOC model, the first NOC model can be assigned with higher weight than the second NOC model. In some embodiments, a NOC model that provides a more accurate prediction under certain circumstances may not provide as accurate prediction under other circumstances. Many variables may affect the accuracy of a particular model, including, for example, the equipment variations, the NOC model's training status, the NOC model's past performance of prediction under similar or different circumstances, the features being selected for a model, etc. As a result, relying on a single model for predicting NOC under all circumstances may not produce accurate or acceptable results. Using a combination of multiple NOC models with assigned weights can reduce the likelihood of a mistaken or inaccurate prediction.

In some embodiments, the weights assigned to each NOC model also reflect preferences or experience of a user. For example, if a user decides that, from the past experience, a particular NOC model is the most trustworthy, a higher weight can be assigned to that particular NOC model. Weights can be any desired numbers that collectively reflect the estimated or assessed capabilities to produce accurate NOC predictions by various NOC models. In one example, the weights assigned to a peak count distribution model, an ANN model, a decision tree model are 0.9, 0.2, and 0.5. In this example, the peak count distribution model is assessed to produce a more accurate NOC prediction than the other two models and is therefore assigned a higher weight. If the NOC estimations are different among different models, the estimation produced by peak count distribution model may be trusted more than the other models.

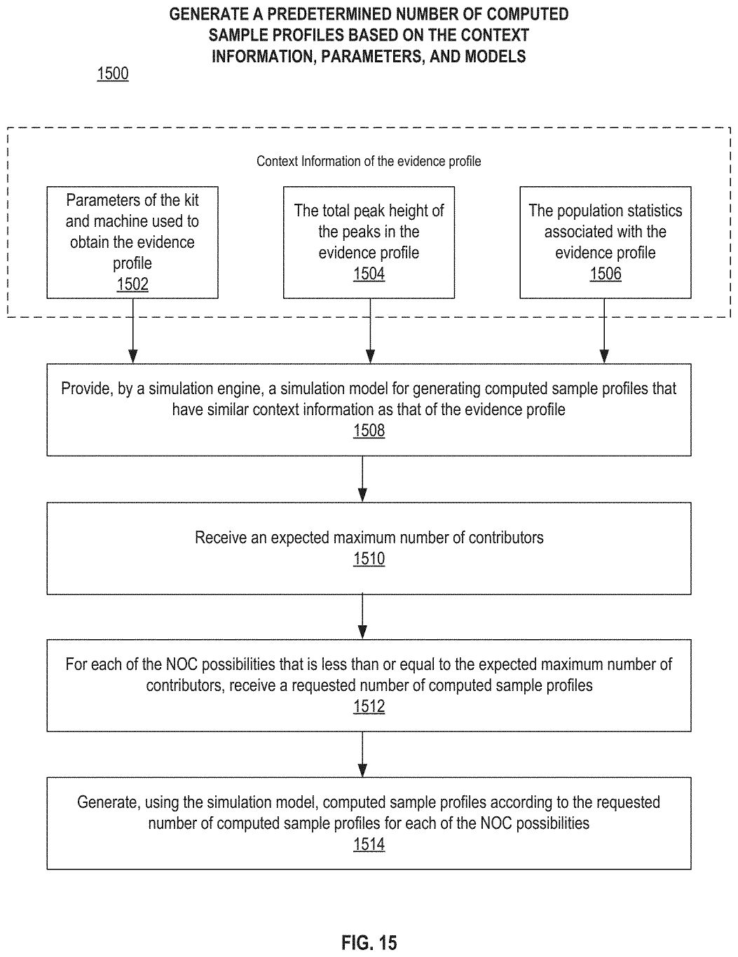

With reference still to FIG. 14, in some embodiments, method 1400 proceeds to obtain context information from the evidence profile in step 1408. Referencing FIG. 15, such context information includes, for example, parameters 1502, a total peak height 1504, and population statistics 1506. Parameters 1502 are associated with a testing kit and/or testing machine used for obtaining the evidence profile. Different testing kit and/or testing machine may result in different models and statistics. These models and statistics may include one or more of a stutter model, a PHR model, an ILB model, a noise model, or the like. Thus, with the knowledge of the specific testing kit and/or machine used for the particular biological sample from which the evidence profile is obtained, proper statistic models can be generated, or selected from a model/statistics database, for using in the subsequent generation of computed sample profiles.

Total peak height 1504 is the sum of the peak heights of the peaks in the evidence profile. The total peak height of the peaks in the evidence profile is one type of context information that is used in generating the computed sample profiles. The total peak height represents the total signal intensity and relates to the amount of DNA contained in the biological sample from which the evidence profile is obtained.

In some embodiments, population statistics 1506 is associated with the evidence profile and obtained as a part of the context information. Different contributors of the biological sample may be from different populations. Thus, for a particular evidence profile, the genotypes corresponding to the contributors may comprise fewer genotypes than a total number of genotypes potentially associated with a current locus in the general population. For example, certain alleles may be common among a large population (e.g., 80% of the general population) while other alleles may be only seen in a smaller population (e.g., 2% of the general population). Further, population statistics may also include models that take into account of shared ancestry in a population. Thus, for different evidence profiles obtained from different biological samples having different contributors, proper population statistics are obtained for using in the subsequent generation of computed sample profiles.

Referencing back to FIG. 14, based on the context information, parameters, and models, step 1410 of method 1400 generates computed sample profiles, sometimes also referred to as simulated sample profiles. When using NOC models to estimate the NOC probabilities in a more accurate manner, computed sample profiles are needed to provide sufficient training of NOC models and/or to provide enough data points for obtaining reasonably accurate statistical distributions for NOC estimation. Oftentimes, data obtainable from real biological samples are limited and are not always available in a large quantity. Thus, real sample profiles may not be sufficient to train machine-learning based NOC models and/or to generate statistically significant distributions. To mitigate the insufficient data available from the real sample profiles, simulations can be performed to generate computed sample profiles.

FIG. 15 illustrates a method 1500 for generating computed sample profiles based on context information, parameters, and/or models. Method 1500 can be used to implement at least step 1410 of method 1400 in FIG. 14. In some embodiments, for generating the computed sample profiles, a simulation engine establishes, updates, and/or uses a simulation model. Step 1508 of method 1500 provides various context information, parameters, and models to the simulation engine to establish or update the simulation model for generating the computed sample profiles. For example, step 1508 can provide to the simulation engine context information including, for example, distributions related to computing stutter peaks (forward stutter peaks, backward stutter peaks, half stutter peaks), noise mode defined for each locus, shoulders, inter locus balance (ILB), the total peak height (representing the total amount of DNA in the evidence profile), peak height ratio (PHR), population frequencies and decay (for each locus and each allele), or the like. The simulation engine establishes and/or updates the simulation model using the context information. For instance, the simulation engine can establish and/or updates the simulation model by obtaining certain available profiles (e.g., real sample profiles or previously computed profiles) and applying parameter variations to these available profiles. These parameter variations can be predefined to be in certain boundaries. For example, if the simulation engine varies the allele peak locations, the variation is limited to those possible or likely allele numbers for any particular locus. As another example, if the simulation engine varies the total peak heights and allocates peak heights to each peaks in a computed profile, the total peak height variation may be limited within a certain percentage from that of the evidence profile. By defining variation ranges of the parameters, the simulation engine can establish and/or update the simulation model such that it generates computed profiles resembling the evidence profile. In some embodiments, the simulation model uses the same or similar distributions (e.g., normal distribution, Gamma distribution, Log normal distribution) that are also used to define the statistical models of certain context information. For instance, if the statistical model related to stutter peaks is defined using a normal distribution, the simulation model can use the same distribution to generate simulated stutter peaks having the same normal distribution. Similarly, the simulation model can use a statistical model related to noise to generate simulated noise having the same distribution. It is understood that the simulation model can be configured with any desired rules, policies, limits, or the like.

Using the simulation model, the simulation engine generates computed sample profiles having context information that resembles that of the evidence profile. For example, the computed sample profiles may have the total peak height (which reflects the total amount of DNA) that is the same or similar to that of the evidence profile (e.g., within a certain predetermined percentage of variation). The computed sample profiles may also have the population statistics and stutter distributions that are similar to those of the evidence profile. As another example, if a biological sample from which the evidence profile is obtained has one or more known contributors, the computed sample profiles can be generated while taking into account known peaks of the one or more known contributors. When generating the computed sample profiles, the simulation engine generates simulated peaks for all the unknown contributors and includes known peaks of the one or more known contributors in the computed sample profiles. Depending on the contribution ratio scenarios, the known peaks of the one or more known contributors may have different peak heights in different computed sample profiles. In some embodiments, the known peaks of the one or more known contributors may be allele peaks and the simulation engine can generate simulated stutter peaks, noise, or the like. As a result, the computed sample profiles and the evidence profile share at least some commonalities with respect to the context information. The commonalities between the evidence profile and the computed profiles facilitate improving the accuracy of the NOC prediction process, because the computed sample profiles are used in subsequent training of machine-learning based NOC models and/or in obtaining statistical distributions for predicting the NOC.

In some embodiments, the simulation engine establishes and/or updates the simulation model based on a set of samples from which one or more evidence profiles are obtained. In one embodiment, the simulation model is established or updated by using default settings from a particular laboratory. The default settings may be adjusted to account for the context information of a particular evidence profile if there are differences of some loci or some alleles in the evidence profile from those of the default settings.

In some embodiments, step 1510 of method 1500 receives a user input providing an expected maximum number of contributors. The expected maximum number of contributors can be used to establish an upper boundary of the NOC possibilities. For example, if the user has information to believe that the biological sample have contributions from the victim, the suspect, and no more than two or three witnesses, then the user can specify that the possible number of contributors is no greater than five. The expected maximum number of contributors can limit the range of possible NOCs. In turn, this reduces the requirements for the quantity of the computed sample profiles to be generated, reduces the computational efforts, and improves the likelihood of providing an accurate NOC prediction. The user input providing an expected maximum number of contributors can be a part of parameters 316 (shown in FIG. 3A) received via a user interface as described above.

In step 1512 of method 1500, for each possible number of contributors that is less than or equal to the expected maximum number of contributors, one or more user inputs providing a requested number of computed sample profiles to be generated are received. For instance, if the expected maximum number of contributors is 3, the possible number of contributors may then be 1, 2, or 3. For each of the possible number of contributors, the user input specifies the requested number of computed sample profiles to be generated. For example, the user input may specify that for each of the possible number of contributors 1, 2, or 3, 1000 computed sample profiles should be generated. Therefore, the total number of computed sample profiles is 3000. As another example, the user input may specify different numbers (e.g., 500, 1000, 1500) of computed sample profiles to be generated for different possible number of contributors (e.g., 1, 2, and 3). The user input providing the requested number of computed sample profiles to be generated can be a part of parameters 316 (shown in FIG. 3A) received via a user interface as described above.

In some embodiments, the requested number of computed sample profiles to be generated may need to satisfy a threshold number. For example, the threshold number may be 500. Thus, if the user input specifies a number that is less than 500, the threshold number or a default number may be used instead. Using a threshold number reduces the likelihood that the user input may specify a number that is too low such that there is an insufficient quantity of computed sample profiles for performing subsequent training of models or for obtaining statistically significant distributions for NOC prediction.

Based on the requested number of computed sample profiles, step 1514 of method 1500 generates the computed sample profiles in the requested quantity. The computed sample profiles are simulated profiles generated by using the simulation model. As described above, the simulation engine can generate computed sample profiles having context information that resembles that of the evidence profile. As a result, these simulated profiles and the evidence profile have at least some commonalities with respect to the context information. For example, these simulated profiles may have the same or similar total peak height, which represents the total amount of DNA, as that of the evidence profile. The simulated profiles may have the same or similar stutter peak statistics as the evidence profile. The simulated profiles may have allele peaks that have the same or similar population statistics as the evidence profile. In one embodiment, the simulated profiles are generated such that their context information are as close to the evidence profile as possible. As a result, the computed sample profiles can be used as if they were profiles obtained from real biological samples. These computed sample profiles can thus be used for subsequent processing (e.g., training of a machine-learning based model) to improve the accuracy of NOC prediction.

With reference back to FIG. 14, as described above, step 1404 of method 1400 receives an indication of the selected NOC models (e.g., two or more of an ANN model, a decision tree model, a random forest model, and a peak distribution model). For each of the selected NOC model, step 1412 of method 1400 determines if the model is a machine-learning based model. If the answer is yes, method 1400 proceeds to step 1416. For example, if the user selects the ANN model, the decision tree model, and/or the random forest model, method 1400 determines that these are machine-learning based NOC models. Based on such a determination, step 1416 of method 1400 forms a training dataset, a testing dataset, and a validation dataset using the generated computed sample profiles. The computed sample profiles can be divided in any desired manner to form the datasets. For example, 1000 computed sample profiles can be divided into 400, 300, and 300 for the training dataset, the testing dataset, and the validation dataset, respectively. These datasets can thus be used to train, or update the training of, a selected machine-learning based NOC model. In some embodiments, the quantity of the computed sample profiles is a number (e.g., 1000 or more) that is sufficiently large to provide the training, testing, and validation datasets and sufficient large such that the machine-learning based NOC model can be trained enough to provide an accurate NOC prediction.

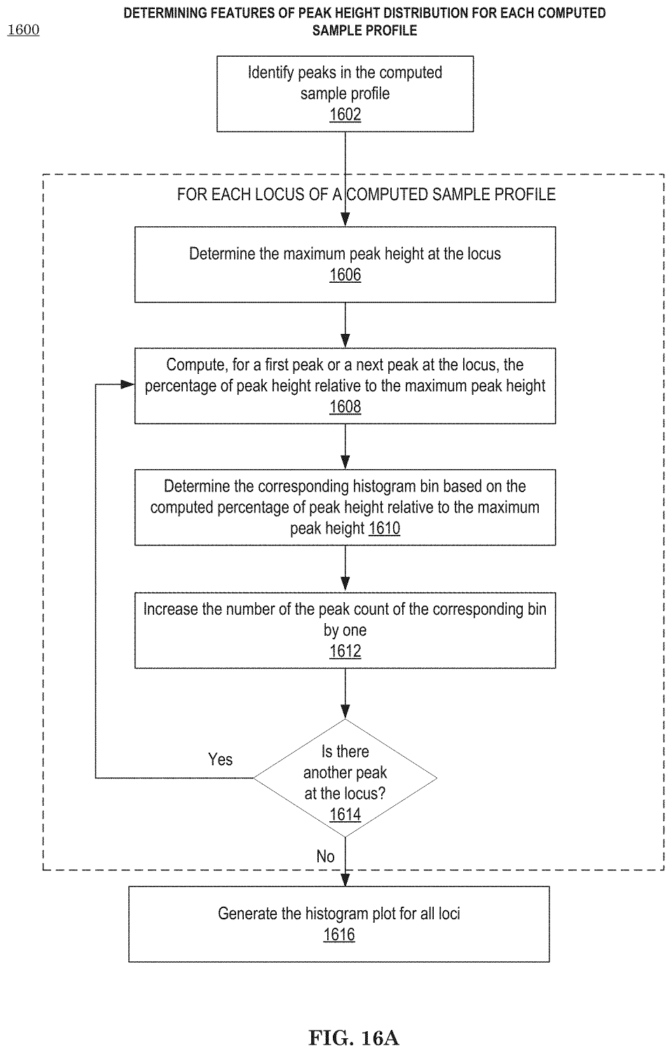

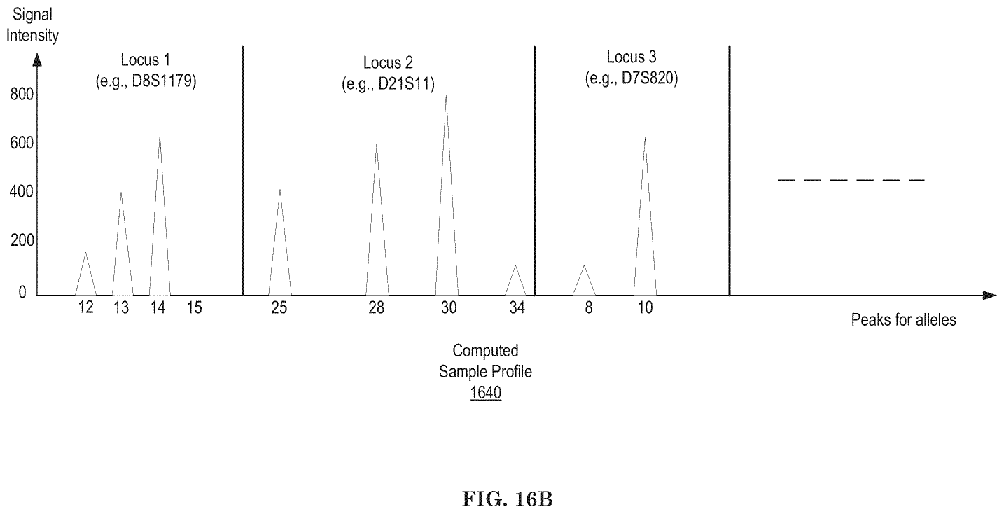

Referencing FIG. 14, in some embodiments, for training a selected machine learning based NOC model, step 1418 of method 1400 determines one or more features of the computed sample profiles. The determination of the features is described in greater detail below using FIGS. 16A, 16B, 17A, 17B and 18 below. Step 1420 trains, or updates the training of, the selected machine-learning based NOC models using the features determined from the computed sample profiles. The training of the selected NOC models can be important because it affects the accuracy of the NOC prediction. Sufficient training data is often required to avoid overfitting. In this disclosure, a desired quantity of computed sample profiles is generated, and features are extracted from these computed sample profiles. As described above, the computed sample profiles are simulated profiles that resemble real sample profiles. Thus, if there is an insufficient number of real sample profiles, the computed sample profiles can be used provide sufficient training data for training the selected machine-learning based NOC models. In some embodiments, the real sample profile and computed sample profiles are combined to provide sufficient training data for training the selected machine-learning based NOC models. Using the training data, the training of the models can be performed followed by testing and validation. In some embodiments, the computed sample profiles are provided for updating the previously trained model. For example, if the context information associated with a new evidence profile is different from that used for training a NOC model, the training can be updated using computed sample profiles generated based on the new evidence profile. Computed sample profiles for the new evidence profile can be generated in a similar manner as described above based on the changed context information.

As shown in FIG. 14, after each of the selected NOC models is trained, or updated, using features extracted from the computed sample profiles, step 1422 of method 1400 estimates the probabilities of multiple NOC possibilities using the trained (or training updated) machine-learning based NOC models. For instance, an ANN model may have one input layer, one or more hidden layers, and one output layer. The ANN model may have any desired number of neurons in the hidden layer(s). The output layer of the trained ANN model provides probabilities of several NOC possibilities up to the expected maximum number of contributors. If the expected maximum number of contributors is, for example, three, the trained ANN model provides the probabilities that the biological sample from which the evidence profile is obtained has 1, 2, or 3 contributors (e.g., 24%, 76%, 0% respectively). Other trained machine-learning based models can similarly provide the probabilities of multiple NOC possibilities.

As described above, for each of the selected NOC model, step 1412 of method 1400 determines if the model is a machine-learning based NOC model. If the answer is no, method 1400 proceeds to step 1414. Step 1414 further determines if the selected NOC model is the peak count distribution model. A peak count distribution model is a statistical model that can be used to predict the NOC based on comparing the total number of peaks to peak count distributions of possible numbers of contributors derived from the large quantity of computed sample profiles. In method 1400, if the answer to the determination in step 1414 is yes, method 1400 proceeds to step 1424. Steps 1424, 1426, 1428, and 1430 are used to perform NOC prediction based on the peak count distribution model.

As described above, when generating the computed sample profiles, method 1400 receives an expected maximum number of contributors (e.g., 3), which may represent the user's expectation or estimation that the NOC cannot be more than the maximum number. Thus, for each possible number of contributors that is less than or equal to the expected maximum number of contributors, step 1424 counts the number of peaks in each of the computed sample profiles. As described above, the computed sample profiles may be generated such that they include a first set of profiles corresponding to one contributor (e.g., 1000 profiles), a second set of profiles corresponding to two contributors (e.g., 1000 profiles), and so forth. Therefore, in each set of these profiles, step 1424 can count the total number of peaks in each of the computed sample profiles. For example, using the first set of profiles corresponding to one contributor, step 1424 may count the total number of peaks to be 45, 50, 55, and 60 for variously different profiles. In the second set of profiles corresponding to two contributors, step 1424 may count the total number of peaks to be 65, 70, 75, and 79 for variously different profiles. In this manner, it is possible to determine peak counts for the different profile sets and account for all possible number of contributors.

When counting the number of peaks, step 1424 of method 1400 may need to distinguish signal peaks from noise. As described in more detail below, in some embodiments, method 1400 distinguishes signal peaks from noise by using a signal intensity threshold (also referred to as the peak height threshold). If the signal intensity (or peak height) of the data at a particular location of the profile is less than the signal intensity threshold, it is likely noise, rather than signal. Thus, in some embodiments, step 1424 identifies the signal peaks before it counts the number of signal peaks.

Based on the number of peaks for each computed sample profile having a corresponding possible number of contributors, step 1426 of method 1400 determines an expected peak count distribution. As described above, step 1424 determines the peak counts in each profile in different sets of profiles corresponding to all possible number of contributors. Based on these peak counts, the number of computed sample profiles that have a certain number of peak counts can be obtained. For instance, for the first set of profiles corresponding to one contributor, step 426 may determine that there are about 80 profiles having peak counts of about 45; about 350 profiles having peak counts of about 50; about 300 profiles having counts of about 55; etc. Similarly, for the second set of profiles corresponding to two contributors, step 1426 may determine that there are about 100 profiles have peak counts of about 65; about 380 profiles having peak counts of about 70; about 100 profiles have peak counts of about 75; etc. The expected peak count distributions for all possible numbers of contributors can thus be determined in a similar manner.

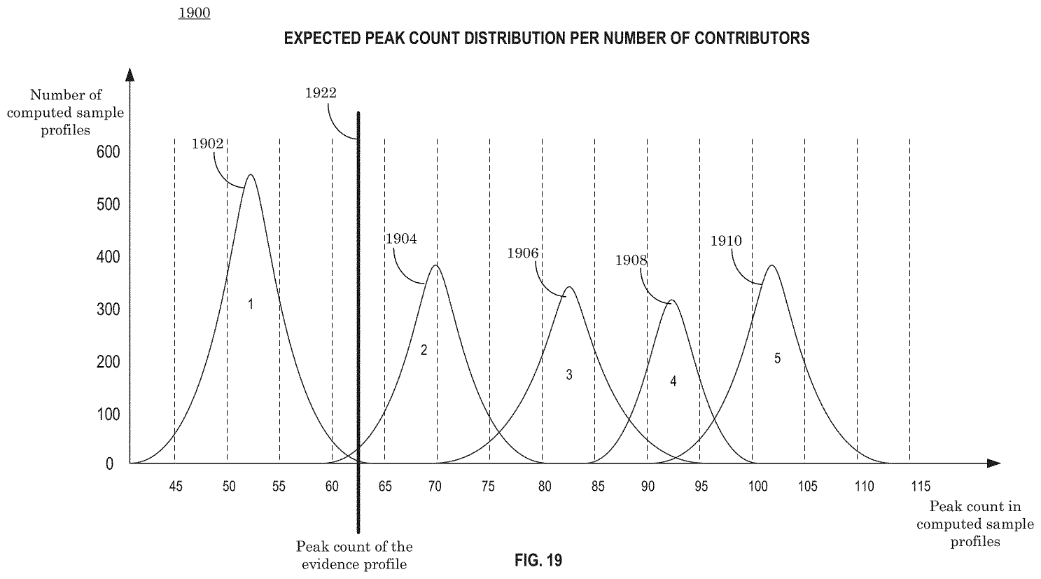

FIG. 19 illustrates example expected peak count distributions for multiple possible number of contributors. In FIG. 19, horizontal axis represents the peak count in computed sample profiles and the vertical axis represents the number of computed sample profiles. Curve 1902 in FIG. 19 is an example expected peak count distribution associated with profiles having one contributor. Curve 1904 is an example expected peak count distribution associated with profiles having two contributors. Curve 1906 is an example expected peak count distribution associated with profiles having three contributors, and so forth. These curves of the expected peak count distributions provide the relation between the numbers of profiles relative to the peak counts in each set of computed sample profiles corresponding to a respective number of contributors. For instance, curve 1902 indicates that, for profiles corresponding to one contributor, the peak counts spread between about 10 to about 65; and a majority number of the profiles has peak counts between about 45 to 60. Similarly, curve 1904 indicates that, for profiles corresponding to two contributors, the peak counts spread between about 60 to 80; and a majority number of the profiles has peak counts between about 65 to 75. Similar indications can be obtained for other curves in FIG. 19.

With reference back to FIG. 14, step 1428 counts the total number of peaks in the evidence profile. Based on the expected peak count distribution and the total number of peaks in the evidence profile, step 1430 estimates the probabilities of multiple NOC possibilities. For instance, as shown in FIG. 19, step 1428 of method 1400 may determine that an evidence profile has a total peak count of about 62, indicated by line 922. This peak count is compared to the peak distributions (e.g., curves 902, 904, 906, 908, and 910). Based on the comparison results, the probabilities that the evidence profile has 1, 2, 3, 4, and 5 contributors are estimated. In the example shown in FIG. 19, because the total peak count of the evidence profile is between the peak count distributions of one contributor and two contributors, it is unlikely that the evidence profile in this example has more than two contributors. Thus, the probabilities that the evidence profile has 3-5 contributors are estimated to be almost zero. The probabilities that evidence profile has one or two contributors may be estimated to be, for example, 40% and 60% respectively.

Referencing back to FIG. 14, if step 1414 determines that the selected NOC model is not the peak distribution model, it proceeds to step 434 to estimate the probabilities of NOC possibilities using any other desired models. These models may include, for example, a maximum allele count (MAC) based model, a heuristic rule model based on an expert, and/or a model based on performing deconvolution analysis. The MAC based model uses MAC distributions extracted from the computed sample profiles. The heuristic rule mode is a type of customized decision tree model that is defined by an expert. For example, a rule defined by the expert may specify that if there is a particular number of peaks (e.g., 3) in a particular locus (e.g., D8S1179), then certain actions should be performed (e.g., a particular path in the decision tree should be followed or a particular classification should result). The model based on performing deconvolution analysis can be used to generate scores using the deconvolution analysis results for all possible numbers of contributors. The scores can be used to predict the NOC.

As described above, probabilities of multiple NOC possibilities (e.g., 1, 2, 3, etc. contributors) can be estimated using machine-learning based NOC models (step 1422), the peak count distribution mode (step 1430), and/or other non-machine learning based models (step 1434). Because multiple selected NOC models are used to estimate the probabilities, their estimation results may or may not have the same or similar probabilities. In some embodiments, method 400 can perform an evaluation of the NOC probabilities provided by multiple NOC models to generate a NOC prediction.

In FIG. 14, steps 1422, 1430, and/or 1434 estimate probabilities of multiple NOC possibilities. These estimated probabilities are provided to step 1432 for evaluation. To evaluate the estimated probabilities provided by different selected NOC models, step 1432 computes, for each selected model, a multiplication of the weight assigned to the selected model and the estimated probabilities provided by the particular selected model. The multiplication results are referred to as scaled probabilities. As described above, in step 1406, weights can be obtained for selected NOC models. The weights can be assigned based on user inputs, default settings, past prediction results, etc. As one example, the weights assigned to a peak count distribution model, an ANN model, a decision tree model are 0.9, 0.2, and 0.5 respectively. For each model, each estimated probability of multiple NOC possibilities is multiplied by the corresponding weight assigned to the particular selected NOC model. For instance, one of the selected NOC models may be the peak count distribution model and may be assigned a weight of 0.9. For this peak count distribution model, the estimated probabilities of the multiple NOC possibilities are, for example: 40% for one contributor, 50% for two contributors, 10% for three contributors, and 0% for four or more contributors. To obtain the scaled probabilities (also referred to as the weighted probabilities), the weight of 0.9 is multiplied to these probabilities. As a result, the scaled probabilities are 36% for one contributor, 45% for two contributors, 9% for three contributors, and 0% for four or more contributors. The scaled probabilities of other selected NOC models can be computed in a similar manner. For example, the ANN model may estimate the probabilities of the multiple NOC possibilities to be: 70% for one contributor, 20% for two contributors, 10% for three contributors, and 0% for four or more contributors. If the ANN model has an assigned weight of 0.2, the scaled probabilities are thus 14% for one contributor, 4% for two contributors, 2% for three contributors, and 0% for four or more contributors. As another example, the decision tree model may estimate the probabilities of the multiple NOC possibilities to be 20% for one contributor, 60% for two contributors, 20% for three contributors, and 0% for four or more contributors. If the decision tree model has an assigned weight of 0.5, the scaled probabilities are thus 10% for one contributor, 30% for two contributors, 10% for three contributors, and 0% for four or more contributors.

Next, for each NOC possibility of the plurality of NOC possibilities, step 1436 of method 400 computes a weighted sum using the scaled probabilities of the multiple selected models. Using the above example, if the peak count distribution model, the ANN model, and the decision tree model are the selected models, the weighted sum for one contributor can be computed by summing the scaled probabilities for one contributor across all three models. Continuing with the above example, the weighted sum for one contributor across all three models is computed to be 60% (i.e., 36%+14%+10%). Similarly, the weighted sum for two contributors is computed to be 79% (i.e., 45%+4%+30%). And the weighted sum for three contributors is computed to be 21% (i.e., 9%+2%+10%).

In some embodiments, step 436 may also compute a normalized weighted sum, which is obtained by dividing the weighted sums of the scaled probabilities by the sum of the weights assigned to all selected models. Continuing with the above example, the sum of the weights assigned to the peak count distribution model, the ANN model, and the decision tree model is 1.6 (i.e., 0.9+0.2+0.5). Thus, the normalized sums of the scaled probabilities for one, two, and three contributor(s) across all three models are 37.5% (i.e., 60% divided by 1.6), 49.4% (i.e., 79% divided by 1.6), and 13.1% (i.e., 21% divided by 1.6), respectively.

Based on the weighted sums or normalized weighted sums, step 1440 of method 1400 predicts the number of contributors associated with the biological sample from which the evidence profile is obtained. Continuing with the above example, step 1440 can rank the weighted sums or the normalized weighted sums for all possible numbers of contributors in a descending order and/or determine the maximum value. In the above example, the weighed sum for the two contributors is the highest value (i.e., 79% or 49.4 if normalized) among the weighted sums for all possible numbers of contributors. Therefore, method 1400 may predict that the biological sample from which the evidence profile is obtained most likely has two contributors.

In the above example, the peak distribution model has a moderately higher probability estimation for two contributors (50%) than that for one contributor (40%). And the ANN model has a much higher probability estimation for one contributor (70%) than that for two contributors (20%). Therefore, the two models do not provide the same or similar estimations. By using the above-described NOC evaluation process, which takes into account the weight assigned to each NOC model, the estimation differences between different NOC models can be resolved and the accuracy of the NOC prediction can be improved. In the above example, because the weight assigned to the peak count distribution model is much higher than that assigned to the ANN model, the NOC prediction gives more weight to the probability estimation provided by the peak count distribution model. As such, method 400 may predict the NOC to be the same or similar to that estimated by the peak count distribution model (e.g., two contributors in the above example). Feature determination for machine-learning based models

As described above, for each of the user-selected NOC model, step 1412 of method 1400 determines if the model is a machine-learning based NOC model. If the answer is yes, method 1400 proceeds to step 1416. Step 1416 forms datasets using the computed sample profiles and then proceeds to step 1418 to determine one or more features from the computed sample profiles. A feature is a property of a profile (e.g., a computed sample profile, a real sample profile, or an evidence profile). The features used for training the machine-learning based NOC model can be a default set of features used for training, a set of features used in the past trainings, and/or a set of user-selected features. The features of a profile include features that are directly extractable from the profile and features that are computable or derivable using the profile. The features that are directly extractable from a profile include, for example, the total number of peaks, the total peak height, or the like. The features that are computable or derivable from a profile include, for example, the peak height distribution of the profile, relative peak heights, the number of loci and/or markers that are “N” number peaks (N>2), or the like. Features are often useful in training a machine-learning based NOC model if they are complementary and/or orthogonal to one another. For example, the amount of DNA and the double amount of DNA are not features that are orthogonal because they are essentially the same property of the profile. Features are also often more useful or important if they are properties that have impact on predicting the target variable. For example, for estimating the NOC probabilities, the day of the week or the first letter of the sample name may not have any impact on the estimation. However, the peak height distribution likely has some impact on the estimation. Thus, the peak height distribution feature is more useful.

In some embodiments, the features that are used in training a machine-learning based NOC model are selected based on the past experiments (e.g., the validation data from a past trained model), the context information, and the specific machine-learning based NOC model. For estimating the NOC probabilities, for example, a set of features can be identified such that they likely can provide a good estimation using a reasonable number of computed sample profiles. The identified feature sets also do not result in a large increase in the needed training data; require a more complex neural network; cause unacceptably long training time (e.g., days or weeks); or result in a significant risk of overfitting.

In some embodiments, the features that are used in training a machine-learning based NOC model are selected based on the type of sample. Different features may be used for different types of biological samples (e.g., DNA samples, RNA samples, protein samples, etc.). The same or different features may be used for different machine-learning based NOC models. For instance, the ANN model and the random forest model may use different features such that they may each provide an improved NOC estimation. In some embodiments, the features that are used in training a machine-learning based NOC model are derived from a large quantity of samples that represent many or substantially all possible scenarios. For instance, the samples used for training can be generated corresponding to many or substantially all possible scenarios including different amounts of DNA, different testing kits and/or machines, different population frequencies, etc. As such, the machine-learning model is trained by using these samples that potentially cover substantially all possible scenarios. As a result, the trained machine-learning model can be used for many different types of biological samples and/or samples having various other different context information (e.g., amount of DNA, testing kits/machines, populations, etc.). Such a trained model is sometimes desirable if, for example, updating the model cannot be readily performed; limited device storage is available for storing many different models and data; and/or it is impractical to distribute many different models.

In some embodiments, the features used for training a machine-learning based model include the peak height distribution of a profile, a total number of identified peaks, the total peak height, the number of loci and/or markers that have “N” number of peaks (N>2), the percentage of the number of peaks having peak height below a predetermined peak height threshold relative to the total number of peaks, or the like. Each of these features are described in greater detail below.

FIG. 16A illustrates a method 1600 for determining the features of peak height distribution for each computed sample profile. Method 1600 can be used to implement at least a part of step 1418 of method 1400 shown in FIG. 14. As described above, the feature of peak height distribution can be a useful and important feature that likely has impact on the NOC probabilities estimation. Therefore, the peak height distribution feature is a property of a profile that has some predictive nature. Referencing FIG. 16A, method 1600 can be applied to each computed sample profile (or real sample profile) to determine the peak height distribution feature. For a particular profile, step 1602 of method 1600 identifies peaks in the profile. Identification of the peaks in a profile can be based on a signal intensity threshold. For instance, the intensities of signals at each allele location (or stutter location) in a computed sample profile can be compared to a predetermined signal intensity threshold. If the particular signal intensity is greater than or equal to the signal intensity threshold, a signal peak is identified. Using FIG. 16B as an example, the signal intensity of the signal at allele 13 of locus 1 is about 400 RFU. If the predetermined signal intensity threshold is 50 RFU, then a peak at allele 13 is identified. This process can be performed at all locations of all loci to identify peaks (allele or stutter) by distinguishing signals from noise. The signal intensity of a peak is also referred to as the peak height.

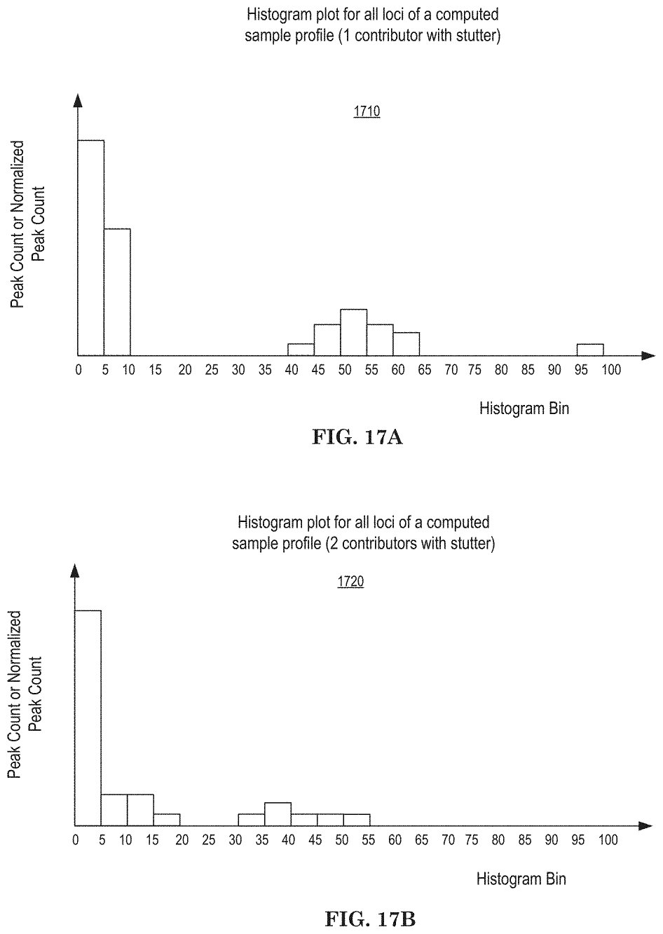

With reference back to FIG. 16A, in some embodiments, for each locus of a computed sample profile, method 1600 performs steps 1606, 1608, 1610, 1612, and 1614. Step 1606 determines the maximum peak height at the locus. For example, for locus 2 in the computed sample profile 1640 shown in FIG. 16B, the maximum peak height can be determined to be about 800 RFU for the peak at allele 30. Next, step 1608 computes, for a first peak or the next peak at the locus, the percentage of peak height relative to the maximum peak height. Continuing with the example in FIG. 16B, at locus 2, step 1608 determines that the peak height of the peak at allele 25 is about 400 RFU, and then computes the percentage of the peak height relative to the maximum peak height (i.e., 50%). Next, step 610 determines the corresponding histogram bin based on the computed percentage of peak height relative to the maximum peak height. A histogram bin can be automatically determined or user selected. In some embodiments, the increment of the histogram bins is predetermined to be 5%. If the horizontal axis of a histogram plot represents the histogram bin numbers, the bin numbers are arranged in 5% increment. FIGS. 17A and 17B show two example histogram plots 710 (FIG. 17A) and 720 (FIG. 17B. In the above example, after step 1608 computes that the percentage of the peak height of the peak at allele 25 is 50%, step 1610 determines that the corresponding histogram bin is bin #50. Next, step 1612 increases the number of the peak count in the corresponding bin by one. Thus, for example, the peak count in the 50% bin of plot 1710 is increased by one. If the percentage of the peak height is a number between two bins (e.g., between bin #45 and bin #50), the percentage of peak height is rounded up or down and the corresponding bin is identified. For instance, if the percentage is 46%, the corresponding bin is then identified to be bin #45. The peak count in bin #45 is then increased by one. Likewise, if the percentage is 58%, the corresponding bin is identified to be bin #60. The peak count in bin #60 is then increased by one.

With reference back to FIG. 16A, step 1614 determines if there is another peak at the locus. If the answer is yes, the process repeats steps 1608, 1610, and 1612. If the answer is no, the process proceeds to step 1616 to generate the histogram data using the identified histogram bins and the corresponding peak counts for all loci of the computed sample profile. Based on the histogram data, histogram plots can be generated. Plot 1710 in FIG. 7A represents an example histogram plot for all loci of a computed sample profile corresponding to one contributor. In plot 1710, the horizontal axis represents the histogram bin numbers and the vertical axis represents the peak counts or normalized peak counts. The profile represented by plot 1710 has one single contributor and accounts for stutter peaks and allele peaks. Stutter peaks tend to have peak heights that are lower (e.g., below 15%) compared to the corresponding allele peaks. Thus, plot 1710 indicates that there are many peaks having small peak heights (e.g., in bin #5 and 10). In plot 1710, the height of the bars represents the peak counts having a certain relative peak height percentage (e.g., 5% of the maximum peak height). For example, in plot 1710, about 60% of all peaks in this computed sample profile are between 0-5% of the peak height. In plot 1710, peak heights of most allele peaks are around 50%. Thus, the profile corresponding to plot 1710 has allele peaks that are roughly the same in peak height (e.g., heterozygous peaks). Typically, a sample from a single person has a profile containing peaks that are roughly the same in peak height. In plot 1710, some peaks have peak height percentage that is around 100% and they are homozygous peaks.

Plot 1720 in FIG. 17B represents another example histogram plot for all loci of another computed sample profile corresponding to two contributors. In plot 1720, the horizontal axis represents the histogram bin numbers and the vertical axis represents the peak counts. The profile represented by plot 1720 has two contributors and accounts for stutter peaks and allele peaks. Similar to plot 1710, plot 1720 indicates that there are many peaks having small peak heights (e.g., in bin #5, 10, and 15). In plot 1720, the heights of the bars represent the peak counts having a certain relative peak height percentage (e.g., 5% of the maximum peak height). Depending on the contribution ratio between the two contributors, the allele peaks may no longer have peak height percentages around 50%. In plot 1720, the values of the peak height percentages spread across more bins (e.g., from bin #5 to 20 and #30 to 55) compared to plot 1710 (e.g., from bin #5 to 10 and 40 to 65). In some embodiments, for a profile that has two or more contributors, fewer peaks are homozygous peaks, as also shown in plot 1720 (e.g., compared to plot 710, there is no bar at the high peak height percentage range corresponding to bin #90-100).

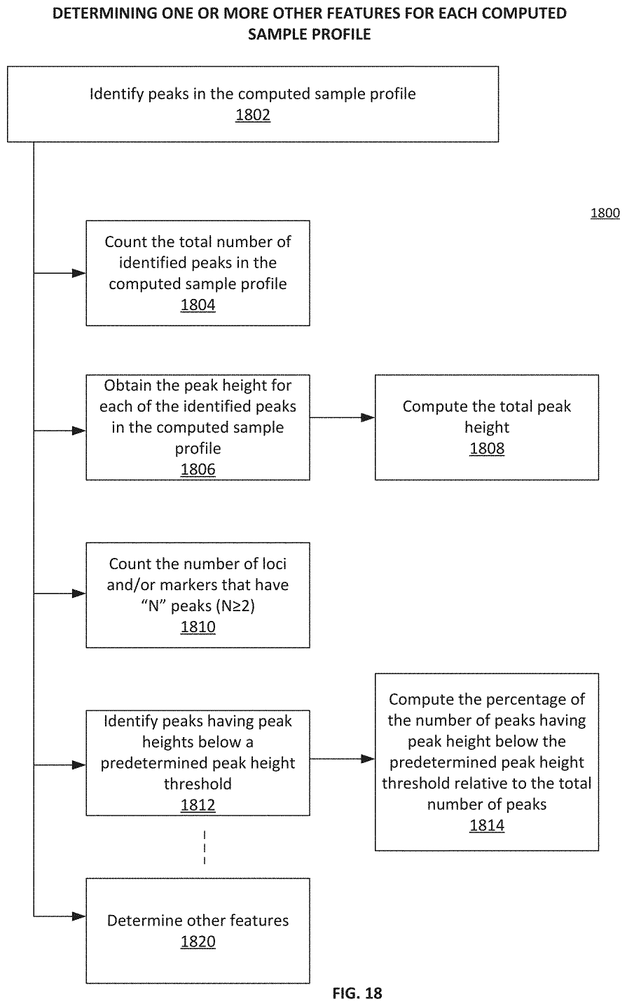

As also indicated by FIG. 17, the peak height distribution of the peaks for all loci in a computed sample profile changes with the number of contributors, and therefore it is a feature that has impact on the NOC probabilities estimation. Other example features that have impact on the NOC probabilities estimation are illustrated in FIG. 18.

FIG. 18 illustrates a method 1800 for determining one or more other features for each computed sample profile. Method 1800 can be used to implement at least a part of step 1418 of method 1400 shown in FIG. 14. Step 1802 of method 1800 identifies peaks in the computed sample profile. Step 1802 is similar to step 1602 of method 1600 and is thus not repeatedly described. In one embodiment, the feature extractable from the computed sample profile is the total number of peaks. Thus, step 1804 counts the total number of identified peaks in the computed sample profile. As described above, the peak identification process distinguishes signal peaks from noise. Therefore, the identified peaks may include allele peaks and stutter peaks, but not noise. Using computed sample profile 1640 shown in FIG. 16B as an example, if profile 1640 includes only three loci as shown, then the total peak count is nine. It is understood that profile 1640 is for illustration of one example. A computed sample profile may include more or fewer peaks and may also have both allele and stutter peaks.

In one embodiment, the feature extractable from a computed sample profile is the total peak height. Based on the identified peaks, step 806 of method 800 obtains the peak height for each of the identified peaks in the computed sample profile. Using computed sample profile 1640 shown in FIG. 16B as an example, if profile 1640 includes only three loci as shown, step 1806 can obtain peak heights of all nine peaks. Next, step 1808 of method 1800 computes the total peak height by summing the peak heights of all peaks in the computed sample profile across all loci. As an example, for profile 1640, the total peak height is computed by summing the peak heights of all nine peaks shown in FIG. 16B. The total peak height of a profile is related to the total amount of DNA in the sample, and thus is a feature that has impact on the NOC probabilities estimation.

In one embodiment, the feature extractable from a computed sample profile is the number of loci and/or markers that have a predetermined number of peaks (e.g., “N” peaks with N>2). In general, if a sample has more contributors, there may be more peaks in some loci. Therefore, the number of loci and/or markers that have a predetermine number has some impact on the NOC probabilities estimation. A locus is a specific fixed position on a chromosome where a particular gene or genetic marker is located. Based on the identified peaks, step 810 of method 800 counts the number of loci and/or markers that have a predetermined number of peaks (e.g., “N” peaks with N>2). As an example, if profile 640 shown in FIG. 6B has only three loci as shown, the number of loci that have three or more peaks is two (i.e., locus 1 and locus 2).

In one embodiment, the feature extractable from a computed sample profile is the percentage of the peaks having peak heights below a predetermined peak height threshold relative to the total number of peaks. As described above using FIG. 17 as an example, in general, if there are two or more contributors, there is a higher number of peaks having smaller peak heights. Thus, the percentage of peaks having smaller peak heights has some impact on the NOC probabilities estimation. As shown in FIG. 18, based on the identified peaks, step 1804 counts a total number of peaks in the computed sample profile. Step 1812 identifies peaks having peak heights below a peak height threshold in the computed sample profile. The peak height threshold may be, for example, 150 RFU. Next, step 1814 computes the percentage of the peaks having peak heights below the predetermined peak height threshold relative to the total number of peaks. Continuing with the example profile 1640 shown in FIG. 16B, there are total of nine peaks in this profile and two peaks (e.g., the peak of allele 34 at locus 2 and the peak of allele 8 at locus 3) have peak heights of about 100 RFU. These two peaks thus have peak heights that are below the predetermined peak height threshold (e.g., 150 RFU). Accordingly, the percentage of the peaks having peak heights below the threshold is computed to be about 22%.

It is understood that FIGS. 16A, 16B, 17A, and 17B illustrate some example features that can be extracted or computed from a profile. Other features can be determined in step 1820 of method 1800. For instance, some features may be specific to a locus or a marker. These features may include, for example, the number of peaks at a particular locus or marker (e.g., marker SE33) and/or the size of the largest peaks for a particular locus or marker (e.g., marker SE33). In some embodiments, the features used in the NOC prediction analysis can be customized based on past analysis results, user input, default settings, and/or any other relevant factors.

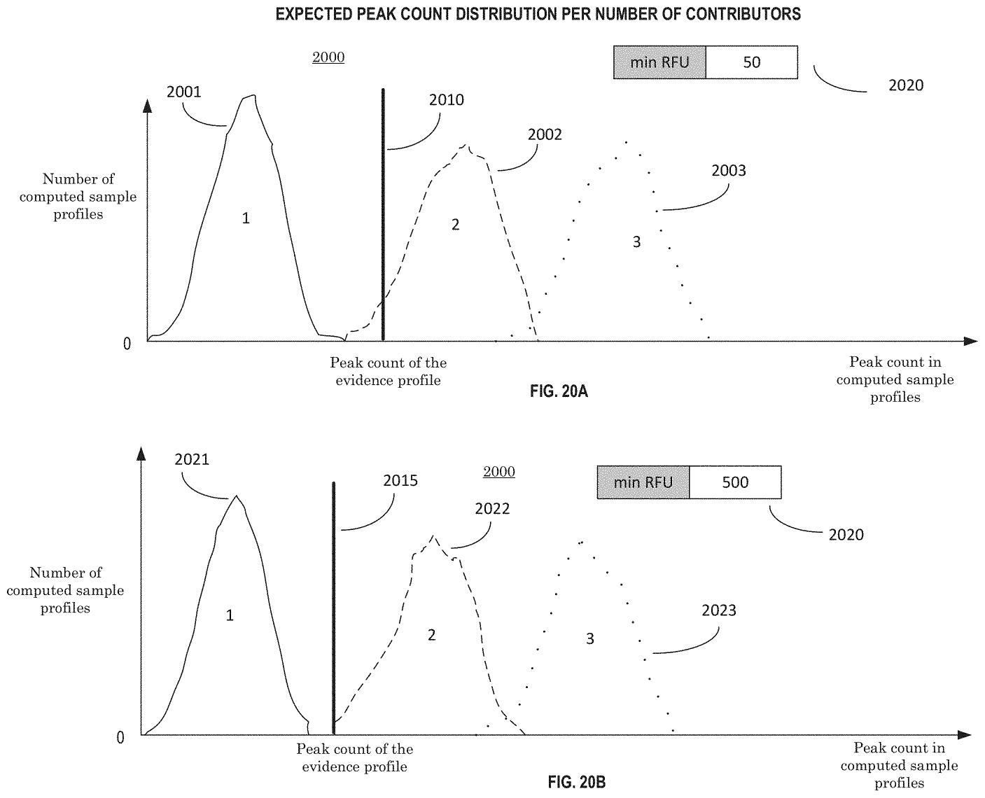

FIGS. 20A-20B illustrate a computer interface 2000 for visualizing impact on peak count distributions of different selections of minimum relative fluorescent units (RFU) for counting peaks in accordance with some embodiments of the present disclosure. As illustrated, interface 2000 allows a user to select a minimum RFU value for peaks in user input area 2020.

In the visual presentation of FIG. 20A, 50 RFU is selected as the minimum value for being counted as a peak in the computed sample profiles and the evidence profile. The expected peak count distribution curves are shown for 1, 2, and 3 contributors scenarios. Specifically, curve 2001 shows an example of an expected peak count distribution of computed profiles having one contributor. Curve 2002 shows an example of an expected peak count distribution of computed profiles having two contributors. Curve 2003 shows an expected peak count distribution of computed profiles having three contributors. Line 2010 shows the total peak count of the evidence profile.

The visual presentation of FIG. 20B shows the expected peak count distributions and the evidence profile if the minimum RFU value for defining a peak to be counted is changed to 500 RFU. Curves 2021, 2022, and 2023 show the expected peak count distributions for one, two, and three contributor profiles, respectively, using 500 RFU, rather than 50 RFU, as the minimum value for purposes of counting a peak. Line 2015 shows the peak count for the evidence profile.

As illustrated in FIG. 20B, the curves have somewhat different shapes than corresponding curves 2001, 2002, and 2003 in FIG. 20A. Also, the distribution curves and the line for the evidence profile have shifted somewhat to the left relative to those shown in FIG. 20A. The visualization of FIG. 20B shows the same overall result as FIG. 20A in the sense that it still suggests that the evidence profile likely reflects 2 contributors but does not likely reflect 3 contributors. At the same time, however, there is some potentially meaningful difference. In FIG. 20B, although the evidence profile (line 2015 in FIG. 20B) is still closer to the two-contributor curve (curve 2022 in FIG. 20B) than it is to the one-contributor curve (curve 2021 in FIG. 20B), it has shifted closer to the one-contributor curve than it was in FIG. 20A (line 2010 and curve 2001 in FIG. 20A), suggesting some increase in the probability that the evidence profile is from one contributor when 500 RFU is set as the minimum value for counting a peak (relative to setting 50 as the minimum RFU for a peak).

An interactive computer interface such as interface 2000 can be helpful for a user who is a forensic expert to visualize and demonstrate whether the choice of minimum RFU value impacts the overall result.

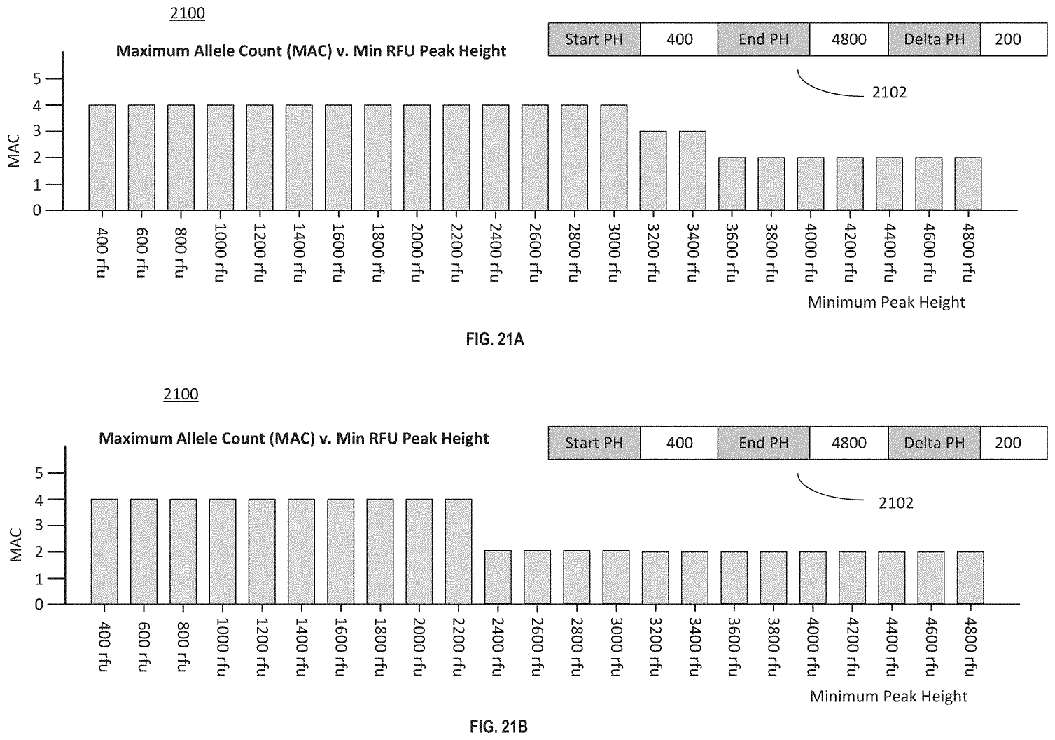

FIGS. 21A-21B show a graphical user interface 2100 for visualizing the relationship between minimum RFU values and maximum allele counts (“MACs”) in different analyzed samples. The MAC value is the largest number of different alleles in any one locus of the analyzed STR loci. In this example, a user can set a range of minimum RFU values for peaks (peak heights or “PH” in the illustration) to visualize the MAC versus peak height. The user can also select the granularity (interval, i.e., “delta,” between peak height values) for the visualization. In these examples, the data is displayed for minimum peak heights from 400 RFU to 4800 RFU, with an interval of 200 RFU. In the illustrated example, the user can change these settings by interacting with user input area 2102.

FIG. 21A shows a computerized visualization of data for a sample with two contributors and a contribution ratio of about 1:3. As shown, the data below 3600 RFUs is consistent with a mixture of two contributors because the MAC values are 4 or 3. For a single contributor, one would expect the maximum allele count to be 2 at any one locus. At peak height cutoffs of 3600 RFU and above, the MAC values are 2. However, this is likely due to the fact that the minor contributor did not generate sufficiently high peaks to have any alleles counted if the minimum peak height to count as an allele is 3600 RFU or higher. Thus the data as a whole is consistent with two contributors and an unequal contribution ratio, and the visualization helps an expert confirm this.

FIG. 21B shows the same visualization for a two-contributor sample but the sample has a different mixture ratio of about 1:7. As shown, alleles for the minor contributor disappear from the data when the minimum RFU for counting a peak is 2400 or higher.

Although the visualizations shown in FIGS. 21A and 21B are not necessarily usable in isolation to definitively determine the number contributors to a mixture or the ratio with which those contributors are represented in the sample, these visualizations in a computerized graphical user interface nevertheless can be useful to an expert in narrowing down a reasonable range of possibilities. For example, although the visualization of the analyzed sample data in FIG. 21B does not by itself definitively show a 1:7 mixture ratio, it is consistent with the minor contributor representing a smaller portion of the sample relative to the major contributor than that shown from the sample visualized in FIG. 21A. Assuming the data is above a range of potential stutter peaks, an earlier (lower RFU value) drop off in MAC from one level to a lower level suggest a smaller ratio between minor and major contributors than does a later (higher RFU value) drop off. Thus the data can help an expert identify which NOC and contribution ratios are more likely than others which can, in turn, be used to speed up deconvolution by prioritizing the more likely scenarios for full deconvolution analysis ahead of less likely scenarios. Moreover, in some examples, such visualizations can help experts identify anomalies in the data and/or confirm the results of other analysis, or help others to better understand the data.

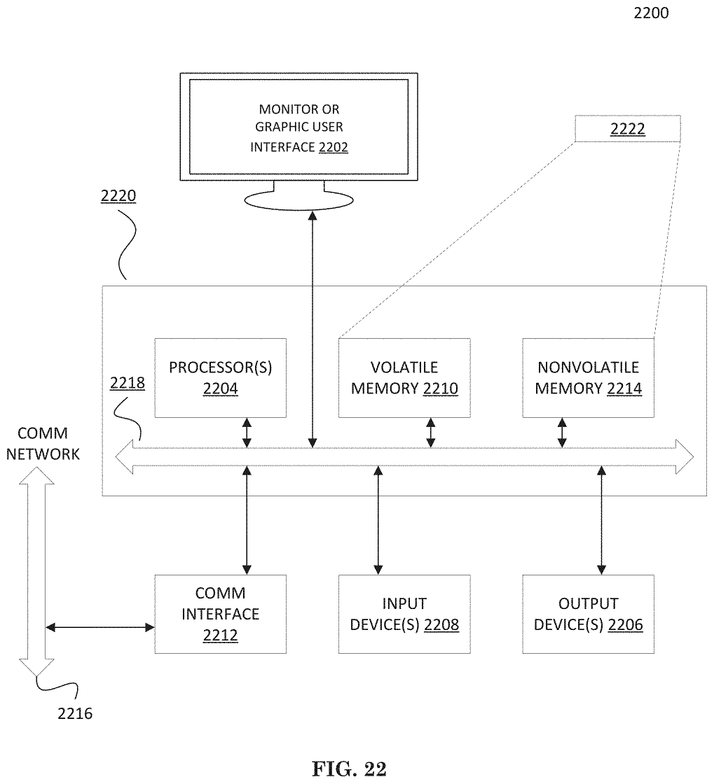

FIG. 22 is an example block diagram of a computing device 1400 that may incorporate embodiments of the present disclosure. FIG. 14 is merely illustrative of a machine system to carry out aspects of the technical processes described herein, and does not limit the scope of the claims. One of ordinary skill in the art would recognize other variations, modifications, and alternatives. In one embodiment, the computing device 1400 typically includes a monitor or graphical user interface 1402, a data processing system 1420, a communication network interface 1412, input device(s) 1408, output device(s) 1406, and the like.

As depicted in FIG. 22, the data processing system 2220 may include one or more processor(s) 2204 that communicate with a number of peripheral devices via a bus subsystem 2218. These peripheral devices may include input device(s) 2208, output device(s) 2206, communication network interface 2212, and a storage subsystem, such as a volatile memory 2210 and a nonvolatile memory 2214. The volatile memory 2210 and/or the nonvolatile memory 2214 may store computer-executable instructions and thus forming logic 2222 that when applied to and executed by the processor(s) 2204 implement embodiments of the processes disclosed herein.

The input device(s) 2208 include devices and mechanisms for inputting information to the data processing system 2220. These may include a keyboard, a keypad, a touch screen incorporated into the monitor or graphical user interface 2202, audio input devices such as voice recognition systems, microphones, and other types of input devices. In various embodiments, the input device(s) 2208 may be embodied as a computer mouse, a trackball, a track pad, a joystick, wireless remote, drawing tablet, voice command system, eye tracking system, and the like. The input device(s) 2208 typically allow a user to select objects, icons, control areas, text and the like that appear on the monitor or graphical user interface 2202 via a command such as a click of a button or the like.

The output device(s) 2206 include devices and mechanisms for outputting information from the data processing system 2220. These may include the monitor or graphical user interface 2202, speakers, printers, infrared LEDs, and so on as well understood in the art.

The communication network interface 2212 provides an interface to communication networks (e.g., communication network 2216) and devices external to the data processing system 2220. The communication network interface 2212 may serve as an interface for receiving data from and transmitting data to other systems. Embodiments of the communication network interface 2212 may include an Ethernet interface, a modem (telephone, satellite, cable, ISDN), (asynchronous) digital subscriber line (DSL), FireWire, USB, a wireless communication interface such as Bluetooth or WiFi, a near field communication wireless interface, a cellular interface, and the like. The communication network interface 2212 may be coupled to the communication network 2216 via an antenna, a cable, or the like. In some embodiments, the communication network interface 2212 may be physically integrated on a circuit board of the data processing system 2220, or in some cases may be implemented in software or firmware, such as “soft modems”, or the like. The computing device 2200 may include logic that enables communications over a network using protocols such as HTTP, TCP/IP, RTP/RTSP, IPX, UDP and the like.

The volatile memory 2210 and the nonvolatile memory 2214 are examples of tangible media configured to store computer readable data and instructions forming logic to implement aspects of the processes described herein. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), battery-backed volatile memories, networked storage devices, and the like. The volatile memory 2210 and the nonvolatile memory 2214 may be configured to store the basic programming and data constructs that provide the functionality of the disclosed processes and other embodiments thereof that fall within the scope of the present disclosure. Logic 2222 that implements embodiments of the present disclosure may be formed by the volatile memory 2210 and/or the nonvolatile memory 2214 storing computer readable instructions. Said instructions may be read from the volatile memory 2210 and/or nonvolatile memory 2214 and executed by the processor(s) 2204. The volatile memory 2210 and the nonvolatile memory 2214 may also provide a repository for storing data used by the logic 2222. The volatile memory 2210 and the nonvolatile memory 2214 may include a number of memories including a main random access memory (RAM) for storage of instructions and data during program execution and a read only memory (ROM) in which read-only non-transitory instructions are stored. The volatile memory 2210 and the nonvolatile memory 2214 may include a file storage subsystem providing persistent (non-volatile) storage for program and data files. The volatile memory 2210 and the nonvolatile memory 2214 may include removable storage systems, such as removable flash memory.

The bus subsystem 2218 provides a mechanism for enabling the various components and subsystems of data processing system 2220 communicate with each other as intended. Although the communication network interface 2212 is depicted schematically as a single bus, some embodiments of the bus subsystem 2218 may utilize multiple distinct busses.

It will be readily apparent to one of ordinary skill in the art that the computing device 2200 may be a device such as a smartphone, a desktop computer, a laptop computer, a rack-mounted computer system, a computer server, or a tablet computer device. As commonly known in the art, the computing device 2200 may be implemented as a collection of multiple networked computing devices. Further, the computing device 2200 will typically include operating system logic (not illustrated) the types and nature of which are well known in the art.

One embodiment of the present disclosure includes systems, methods, and a non-transitory computer readable storage medium or media tangibly storing computer program logic capable of being executed by a computer processor.

Those skilled in the art will appreciate that computer system 2200 illustrates just one example of a system in which a computer program product in accordance with an embodiment of the present disclosure may be implemented. To cite but one example of an alternative embodiment, execution of instructions contained in a computer program product in accordance with an embodiment of the present disclosure may be distributed over multiple computers, such as, for example, over the computers of a distributed computing network.

While the present disclosure has been particularly described with respect to the illustrated embodiments, it will be appreciated that various alterations, modifications and adaptations may be made based on the present disclosure and are intended to be within the scope of the present disclosure. While the disclosure has been described in connection with what are presently considered to be practical embodiments, it is to be understood that the present disclosure is not limited to the disclosed embodiments but, on the contrary, is intended to cover various modifications and equivalent arrangements included within the scope of the underlying principles of the disclosure as described by the various embodiments referenced above and below.

1. A computer-implemented method of selecting subsets of possible allele combinations for further deconvolution analysis to improve computer efficiency in genotyping one or more unidentified contributors of a plurality of contributors to a biological sample using an evidence profile obtained from the biological sample comprising genetic signal data corresponding to short tandem repeat (STR) alleles at each of a plurality of loci, the method comprising, at each locus, for a currently analyzed contribution ratio scenario of a plurality of contribution ratio scenarios, using one or more computer processors to carry out processing comprising:
(a) computing an adjusted evidence profile by subtracting from the evidence profile a computed expected contribution of all known contributors, if any;
(b) for a first, or next, unidentified contributor having a pre-determined highest remaining contribution ratio in the currently analyzed contribution ratio scenario for the plurality of contributors, computing a first range of expected peak heights using at least the pre-determined highest remaining contribution ratio, a selected degradation value, and a peak height ratio distribution;
(c) for all other remaining unidentified contributors, if any, computing a second range of expected peak heights using at least pre-determined contribution ratios in the currently analyzed contribution ratio scenario of the all other remaining unidentified contributors, the selected degradation value, and the peak height ratio distributions; and
(d) using the adjusted evidence profile and one or more of the first range and the second range to select one or more selected genotypes at least potentially corresponding to the first, or next, unidentified contributor for further deconvolution analysis, wherein the one or more selected genotypes comprise fewer genotypes than a total number of genotypes potentially associated with a current locus in a general population.

2. The computer-implemented method of claim 1, wherein (d) comprises selecting an allele twice as required alleles in an allele pair such that the one or more selected genotypes is a single homozygous genotype, if the allele in the adjusted evidence profile has a peak that is at least double of a threshold of the second range.

3. The computer-implemented method of claim 1, wherein (d) comprises selecting an allele as a required allele in an allele pair for each of the one or more selected genotypes if the allele in the adjusted evidence profile has a peak that is above a threshold of the second range and below a double of the threshold of the second range.

4. The computer-implemented method of claim 1, wherein (d) comprises selecting an allele twice as two possible alleles for each of the one or more selected genotypes if the allele in the adjusted evidence profile has a peak that is at least double a threshold of the first range.

5. The computer-implemented method of claim 1, wherein (d) comprises selecting an allele as a possible allele in an allele pair for each of the one or more selected genotypes if the allele in the adjusted evidence profile has a peak that is above a threshold of the first range but below a double of the threshold of the first range.

6. The computer-implemented method of claim 1, further comprising:
based on a predetermined number of contributors of the plurality of contributors and the genetic signal data, determining one or more contribution ratio scenarios representing possible proportions of biological materials contributed by each of the plurality of contributors to the biological sample.

7. The computer implemented method of claim 1, further comprising using one or more computer processors to carry out processing comprising:
for each respective genotype of the one or more selected genotypes at least potentially corresponding to the first unidentified contributor, repeating (a)-(d) for a next unidentified contributor, if any, while treating the first unidentified contributor as a known contributor having a respective genotype of the one or more selected genotypes.

8. The computer implemented method of claim 7, further comprising, using the one or more computer processors to carry out processing comprising, prior to the repeating (a)-(d) for the next unidentified contributor:
determining if a respective genotype of the one or more selected genotypes comprises a pair of required alleles corresponding to the first unidentified contributor.

9. The computer-implemented method of claim 7, further comprising using the one or more computer processors to carry out processing comprising, prior to the repeating (a)-(d) for the next unidentified contributor:
determining if a respective genotype of the one or more selected genotypes comprises only one required allele corresponding to the first unidentified contributor and one or more possible alleles potentially corresponding to the first unidentified contributor; and
if so, computing one or more allele combinations using the one required allele and the one or more possible alleles, wherein the repeating (a)-(d) for the next unidentified contributor is carried out for each of the one or more allele combinations.

10. The computer-implemented method of claim 7, further comprising using one or more computer processors to carry out processing comprising, prior to the repeating (a)-(d) for the next unidentified contributor:
determining if a respective genotype of the one or more selected genotypes comprises only possible alleles potentially corresponding to the first unidentified contributor; and
if so, computing allele combinations using only the possible alleles, wherein repeating the processing of claim 1 for the next unidentified contributor is carried out for each of the allele combinations.

11. The computer-implemented method of claim 7, further comprising using one or more computer processors to carry out processing comprising:
computing a plurality of allele combinations of unidentified contributors by repeating (a)-(d) one or more times until no unidentified contributor remains; and
for each of the plurality of allele combinations of each of the unidentified contributors, computing theoretical contributions to a respective theoretical profile.

12. The computer-implemented method of claim 11, wherein for each of the plurality of allele combinations of each of the unidentified contributors, computing theoretical contributions to a respective theoretical profile comprises, for a respective allele combination of a respective unidentified contributor:
computing allele peaks corresponding to alleles in the respective allele combination, the allele peaks being computed using the currently analyzed contribution ratio scenario for the respective unidentified contributor;
storing the allele peaks for computing a respective theoretical profile;
computing stutter peaks, if any, of at least some of the allele peaks; and
storing the stutter peaks for computing the respective theoretical profile.

13. The computer-implemented method of claim 11, further comprising using one or more computer processors to carry out processing comprising:
computing theoretical contributions corresponding to alleles of the all known contributors, the theoretical contributions having peak heights computed using the currently analyzed contribution ratio scenario;
computing the respective theoretical profile using the theoretical contributions from the unidentified contributors and theoretical contributions from the all known contributors; and
determining a degree of matching between the evidence profile and respective theoretical profile.

14. The computer-implemented method of claim 13, wherein determining a degree of matching between the evidence profile and respective theoretical profile comprises, for each corresponding bin associated with the evidence profile and the respective theoretical profile:
determining a number of alleles in a respective bin;
determining if the number of alleles in the respective bin is greater than zero, and if so, determining one or more probability adjustment parameters; and
computing one or more genotype probabilities based on the one or more probability adjustment parameters and a genotype probability model.

15. The computer-implemented method of claim 14, further comprising:
determining if there is a missing peak in the evidence profile, and if so, computing a probability of dropout; and
determining if there is a missing peak in the theoretical profile, and if so, computing a probability of dropin.

16. The computer-implemented method of claim 15, further comprising:
if there is no missing peak in the theoretical profile and no missing peak in the evidence profile, determining if peak heights in the corresponding bin of the theoretical profile and the evidence profile are greater than a pre-defined threshold;
if so, computing a probability of peak height mismatch between the peaks at the corresponding bin of the theoretical profile and the evidence profile; and
computing a score of the respective bin using one or more of the one or more genotype probabilities, the probability of dropout, the probability of dropin, and the probability of peak height mismatch.

17. The computer-implemented method of claim 16, further comprising:
computing a profile score using the score of all bins associated with the theoretical profile and the evidence profile;
determining, using the profile score, if the degree of matching between the evidence profile and the respective theoretical profile satisfies a matching threshold; and
if so, providing a likelihood of matching between the one or more unidentified contributors and one or more persons-of-interest (POIs).

18. The computer-implemented method of claim 1, wherein (b) comprises:
computing a sum of peak heights of the peaks in the adjusted evidence profile;
computing first expected peak heights associated with the first, or next, unidentified contributor using the sum of peak heights, the pre-determined highest remaining contribution ratio, and the selected degradation value;
adjusting the first expected peak heights using one or more expected stutter peak heights; and
computing the first range of expected peak heights using the adjusted first expected peak heights and the peak height ratio distribution.

19. The computer-implemented method of claim 1, wherein (c) comprises:
computing a sum of peak heights of the peaks in the evidence profile;
computing second expected peak heights associated with the all other remaining unidentified contributors using the sum of peak heights, the at least pre-determined contribution ratios in the currently analyzed contribution ratio scenario of the all other remaining unidentified contributors, and the selected degradation value;
adjusting the one or more second expected peak heights using one or more expected stutter peak heights; and
computing the second range of expected peak heights using the adjusted one or more second expected peak heights and the peak height ratio distribution.

20. A non-transitory computer readable medium storing one or more instructions which, when executed by one or more processors of at least one computing device, perform processing to select subsets of possible allele combinations for further deconvolution analysis to improve computer efficiency in genotyping one or more unidentified contributors of a plurality of contributors to a biological sample using an evidence profile obtained from the biological sample comprising genetic signal data corresponding to short tandem repeat (STR) alleles at each locus of a plurality of loci, the processing comprising, at each locus, for a currently analyzed contribution ratio scenario of a plurality of contribution ratio scenarios:
(a) computing an adjusted evidence profile by subtracting from the evidence profile a computed expected contribution of all known contributors, if any;
(b) for a first, or next, unidentified contributor having a pre-determined highest remaining contribution ratio in the currently analyzed contribution ratio scenario for the plurality of contributors, computing a first range of expected peak heights using at least the pre-determined highest remaining contribution ratio, a selected degradation value, and a peak height ratio distribution;
(c) for all other remaining unidentified contributors, if any, computing a second range of expected peak heights using at least pre-determined contribution ratios in the currently analyzed contribution ratio scenario of the all other remaining unidentified contributors, the selected degradation value, and the peak height ratio distributions; and
(d) using the adjusted evidence profile and one or more of the first range and the second range to select one or more selected genotypes at least potentially corresponding to the first, or next, unidentified contributor for further deconvolution analysis, wherein the one or more selected genotypes comprise fewer genotypes than a total number of genotypes potentially associated with a current locus in a general population.

21-69. (canceled)

70. A computer-implemented method of providing, on an electronic display, a graphical user interface (GUI), the computer-implemented method comprising:
receiving, via the GUI, a user-entered minimum peak height value for fluorescence data corresponding to analyzed alleles at a plurality of loci in a biological sample via the GUI;
using the minimum peak height value to compute and display, in the GUI on the electronic display, a plurality of distribution visual representations, each of the plurality of distribution visual representations showing a distribution of computed sample profiles for an assumed number of contributors for a range of peak counts; and
using the minimum peak height value to compute and display, in the GUI on the electronic display, an evidence profile visual representation showing a peak count of the evidence profile, wherein the evidence profile visual representation is displayed relative to the plurality of distribution visual representations.

71. The computer-implemented method of claim 70 further comprising:
displaying, in the GUI on the electronic display, two or more visual presentations, each visual presentation comprising a plurality of distribution visual representations and an evidence profile visual representations, wherein each of the two or more visual presentations is computed and displayed using a different minimum peak height value.

72. The computer-implemented method of claim 71 wherein the two or more visual representations are displayed relative to each other in a manner to facilitate comparison of a first visual presentation corresponding to a first minimum peak value and a second visual presentation corresponding to a second minimum peak value.

73. The computer-implemented method of claim 72 wherein the two or more visual representations are horizontally aligned and displayed one above another.

74-75. (canceled)
